(19) United States
(12) Patent Application Publication
(10) Pub. No.: US 2005/0070480 A1
(43) Pub. Date: Mar. 31, 2005

(54) Peptide arginals and methods for treating disseminated intravascular coagulation

(76) Inventor: Sandor Bajusz, Budapest (HU)

Correspondence Address: IVAX CORPORATION, 4400 BISCAYNE BOULEVARD, MIAMI, FL, 33137, US

(21) Appl. No.: 10/487,465

(22) PCT Filed: Aug. 21, 2002
(86) PCT No.: PCT/US02/26564

Related U.S. Application Data
(60) Provisional application No. 60/313,850, filed on Aug. 21, 2001.
(60) Provisional application No. 60/313,901, filed on Aug. 21, 2001.

(51) Int. Cl. A61K 38/05; C07K 5/06

(52) U.S. Cl. 514/18; 530/331; 548/537

(57) ABSTRACT

The invention relates to disseminated intravascular coagulation. More particularly, the invention relates to medical intervention disseminated intravascular coagulation. The invention provides new peptides arginals, new and better compounds and methods for the treatment of DIC. The compounds and methods according to the invention have inhibitory action on clot-bound thrombin and factor Xa and are also inhibitory against plasmin and plasminogen activators.

### BACKGROUND OF THE INVENTION

1. Field of the Invention

The invention relates to disseminated intravascular coagulation. More particularly, the invention relates to medical intervention for disseminated intravascular coagulation.

2. Summary of the Related Art

Disseminated intravascular coagulation (DIC) is a secondary disease and can be a consequence of any of a large number of primary diseases. (See Bick, Disseminated Intravascular Coagulation and Related Syndromes, CRC Press, Boca Raton, (1983)). Among its characteristics is the systematic activation of blood coagulation that results in the generation and deposition of fibrin, leading to microvascular thrombi in various organs and contributing to the development of multi-organ failure. Bick et al, Clin. Appl Thrombosis Hemostasis 1: 3-23 (1995) teaches that a further characteristic of DIC is systemically circulating plasmin, a global proteolytic enzyme that can biodegrade various plasma proteins (factors, hormones etc.) and can cleave fibrinogen/fibrin to yield fibrinogen/fibrin degradation products. These products impair hemostasis and lead to hemorrhage.

The most serious clinical form of DIC is characterized by extensive consumption of coagulation proteins, significant deposition of fibrin, and bleeding.

Trauma patients are at increased risk for DIC, especially when there are widespread areas of tissue damage (particularly the brain), sepsis and multiple organ failure. The head trauma is a particularly common cause of DIC in infants and children because of the high thromboplastin content of the brain and the proportionately increased ratio of head surface area to total body surface area.

Sepsis may occur in about 40% of all trauma patients and is an important primary cause of DIC in all patients. The clinical condition is worsened by secondary fibrinolysis, which results in the formation of FDP's (fibrinogen/fibrin degradation products) or “D-dimers” that interfere with normal fibrin formation and platelet function.

Fibrin deposition in DIC may lead to further organ dysfunction. DIC is a major cause of acute renal failure and also contributes to multiple system organ failure. The converse is also true, with the damaged organs contributing to DIC.

Currently, the only accepted treatment for DIC is limited to attempting to alleviate the primary disorder. Without control DIC will continue despite forms of therapy directed at correcting the bleeding or thrombotic problem. In some cases in which there is significant bleeding, replacement therapy with fresh frozen plasma, plasma components (e.g., antithrombin III) cryoprecipitate, and/or platelet concentrates may be helpful until the primary problem is controlled, but these therapies are prohibitively expensive. The use of heparin in DIC is highly controversial and is not generally used in patients with an underlying problem of trauma.

There is, therefore, a need for new and better compounds and methods for the treatment of DIC [See also, e.g., de Jonge et al, Drugs 55: 767-777 (1998) and Levi et. al., Thrombosis and Haemostasis 82: 695 (1999)].

### BRIEF SUMMARY OF THE INVENTION

The invention provides new and better compounds and method for the treatment of DIC. It has been surprisingly found that those anticoagulant compounds that have inhibiting action on both free and clot-bound thrombin and factor Xa and also are inhibitory against plasmin and plasminogen activators can be useful for the treatment of DIC.

In a first aspect, the invention provides a composition of matter comprising a peptidyl arginal of the formula (I)
Xaa-Xbb-Arg-H (I)
wherein Xaa represents an alpha-substituted carbonic acid residue of formula (II)
Q-CH(R)—CO (II)
wherein Q represents a 1-3 carbon alkyloxycarbonylamino group, a methylamino group, or a hydroxyl group, and R represents a 7-9 carbon cycloalkylmethyl group, or a 5-7 carbon cycloalkyl group, or a 1-adamantylmethyl group, and Xbb represents an L-proline or L-azetidine-2-carboxylic acid residue, and the acid-addition salts thereof formed with organic or inorganic acid.

In particularly preferred embodiments such compounds may have the following structures: 1 (ethoxycarbonyl-D-cycloheptylalanyl-L-prolyl-L-arginine aldehyde, Eoc-D-cHpa-Pro-Arg-H), or 2 (N-methyl-D-cycloheptylalanyl-L-prolyl-L-arginine aldehyde, ND-cHpa-Pro-Arg-H), or 3 (D-cycloheptyllactyl-prolyl-L-arginine aldehyde, D-cHpl-Pro-Arg-H), or 4 (N-methyl-D-cyclohexylglycyl-L-azetidine-2-carbonyl-L-arginine aldehyde, N-Me-D-Chg-Aze-Arg-H), which correspond to the formula (I) wherein Xaa represents an alpha-substituted alkyl carbonic acid residues of the formula (I) wherein R represents a cycloheptylmethyl and cyclohexyl group, respectively, Q represents an ethoxycarbonylamino, methylamino, and hydroxyl group, respectively, and Xbb represents L-proline and L-azetidinyl-2-carboxylic acid residue, respectively.

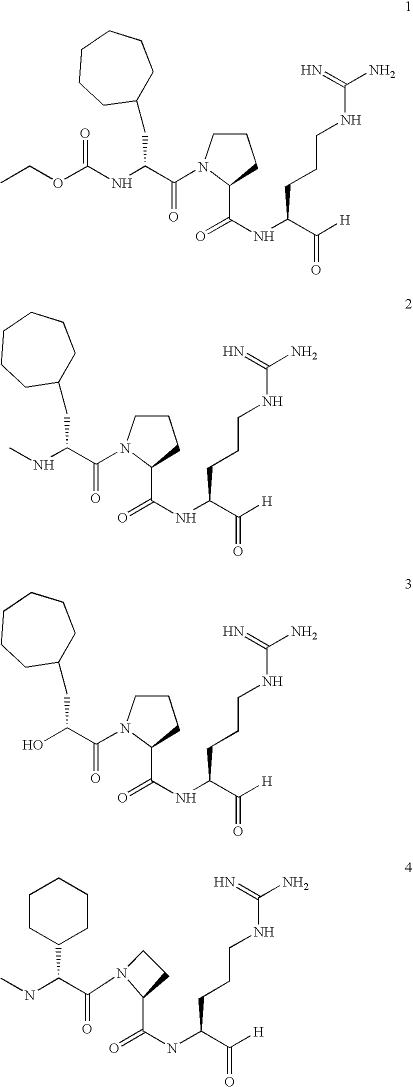

In a second aspect, the invention provides a pharmaceutical composition comprising an anticoagulant peptidyl arginal or a pharmaceutically acceptable salt thereof according to the first aspect of the invention and a pharmaceutically acceptable carrier, excipient or diluent.

In a third aspect, the invention provides a method for treating disseminated intravascular coagulation, the method comprising administering to a patient having disseminated intravascular coagulation an anticoagulant peptidyl arginal corresponding to the formula (I)
Xaa-Xbb-Arg-H (I)
wherein Xaa represents an alpha-substituted carbonic acid residue of form a (II)
Q-CH(R)—CO (II)
wherein Q represents a 1-3 carbon alkyloxycarbonylamino group, a methylamino group, or a hydroxyl group, and R represents a 7-9 carbon cycloalkyl methyl group, or a 1-adamantylmethyl group or a 5-7 carbon cycloalkyl group, and Xbb represents an L-proline or an azetidine-2-carboxylic acid residue, or a pharmaceutically acceptable acid addition salt thereof. In particularly preferred embodiments such compounds may have the following structures 1 (ethoxycarbonyl-D-cycloheptylalanyl-L-prolyl-L-arginine aldehyde, Eoc-D-cHpa-Pro-Arg-H), or 2 (N-methyl-D-cycloheptylalanyl-L-prolyl-L-arginine aldehyde, N-Me-D-cHpa-Pro-Arg-H, or 3 (D-cycloheptyllactyl-L-prolyl-L-arginine aldehyde, D-cHpl-Pro-Arg-H), or 4 (N-methyl-D-cyclohexylglycyl-L-azetidine-2-carbonyl-L-arginine_aldehyde, N-Me-D-Chg-Aze-Arg-H), which correspond to the formula (I) wherein Xaa, represents an alpha-substituted alkyl carbonic acid residues of the formula (II) wherein R represents a cycloheptylmethyl and a cyclohexyl group, respectively, Q represents an ethoxycarbonylamino, methylamino, and a hydroxyl group, respectively, and Xbb represents an L proline and L-azetidinyl-2-carboxylic acid residue, respectively.

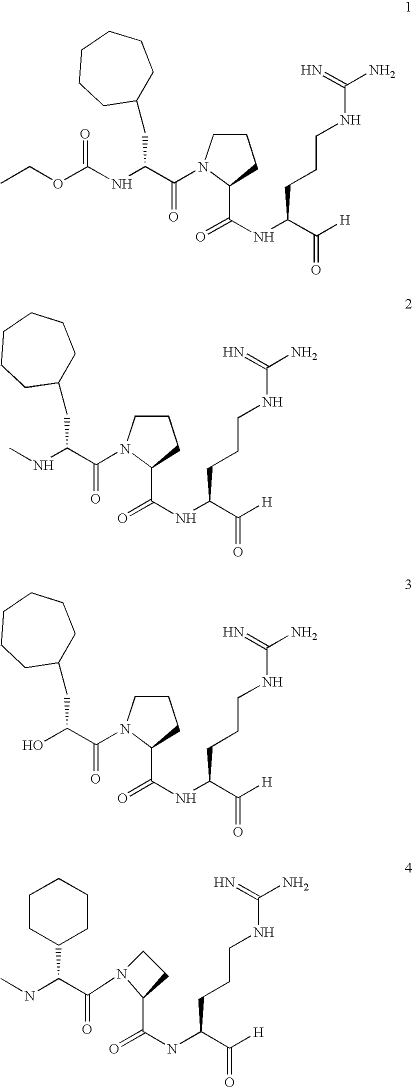

### DETAILED DESCRIPTION OF THE PREFERRED EMBODIMENTS

The invention relates to disseminated intravascular coagulation. More particularly, the invention relates to medical intervention for disseminated intravascular coagulation. The invention provides new and better compounds and method for the treatment of DIC. The compounds according to the invention have inhibitory action on both free and clot-bound thrombin and factor Xa, as well as on plasmin and plasminogen activators.

The patents and publications cited herein reflect the knowledge in the art and are hereby incorporated by reference in entirety. Any inconsistency between these patents and publications and the present disclosure shall be resolved in favor of the present disclosure
n a first aspect, the invention provides a composition of matter comprising a peptidyl arginal having the formula (I)
   Xaa-Xbb-Arg-H (I)
   wherein Xaa represents an alpha-substituted carbonic acid residue of the formula (II)
   Q-CH(R)—CO (II)
   wherein Q represents a 1-3 carbon alkyloxycarbonylamino group, a methylamino group, or a hydroxyl group, and R represents a 7-9 carbon cycloalkylmethyl group, a 1-adamantylmethyl group, or a 5-7 carbon cycloalkyl group, and Xbb represents an L-proline or L-azetidinyl-2-carboxylic acid residue, and the acid addition salts thereof formed with organic or inorganic acid.

A particularly preferred embodiment according to this aspect of the invention corresponds to structure 1 (ethoxycarbonyl-D-cycloheptylalanyl-L-prolyl-L-arginine aldehyde, Eoc-D-cHpa-Pro-Arg-H):

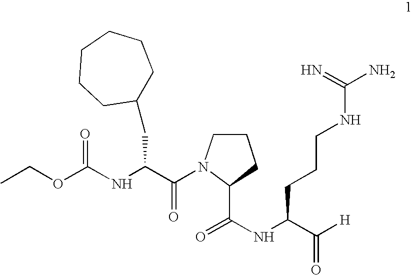

Another particularly preferred embodiment according to this aspect of the invention corresponds to structure 2 (N-methyl-D-cycloheptylalanyl-L-propyl-L-arginine aldehyde, N-Me-D-cHpa-Pro-Arg-H):

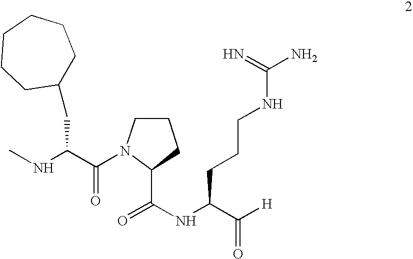

A further particularly preferred embodiment according to this aspect of the invention corresponds to structure 3 (D-cycloheptyllactyl-L-prolyl-L-arginine aldehyde, D-Hpl-Pro-Arg-H):

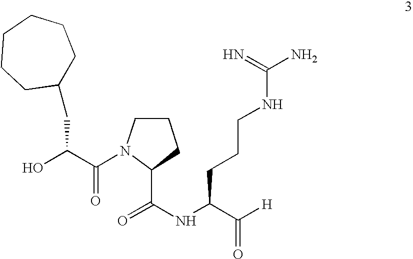

An even further particularly preferred embodiment according to this aspect of the invention corresponds to structure 4 (N-methyl-D-cyclohexylglycyl-L-azetidine-2-carbonyl-L-arginine aldehyde, N-Me-D-cHpa-Pro-Arg-H):

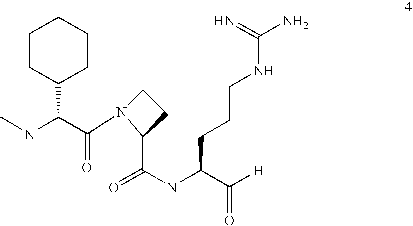

The compounds according to formula 1, 2, 3 and 4 are prepared e.g., by condensing the N or O-protected 2-residue acid component with an L-arginine lactam, protected on the guanidino group with a benzyloxycarbonyl group and reducing the obtained tripeptide lactam to the protected tripeptide aldehyde, removing the protecting group from the guanidino group of arginine, and in case of Q=methylamino or hydroxyl in formula (II) also from the terminal methylamino or hydroxyl group, and isolating the peptide derivative of formula (I) as its addition salt formed with an organic or inorganic acid.

The compounds represented by the formula (I) are prepared and used in the form of acid-addition salts owing to the greater stability of the salt forms. In the acid-addition salts of the compound of formula (I) the activity resides in the base and the acid is of less importance although for therapeutic purposes it is preferable to use pharmaceutically acceptable acid-addition salts. Examples of such suitable acids include (a) mineral acids: hydrochloric, hydrobromic, phosphoric, metaphosphoric and sulphuric acids, (b) organic acids: tartaric, acetic, citric, malic, lactic, fumaric, benzoic, glycolic, gluconic, succinic, pamoic and aryl-sulphonic acids, for example p-toluenesulphonic acid. Preferred acid-addition salt is the sulphate especially the hemisulphate salt.

The acid-additional salts are prepared in a conventional manner e.g. by neutralizing the free base form of the compound of formula (I) with the acid.

The two residue acid component can be shown as D-Xaa-Xbb, wherein Xaa represents an α-substituted carboxylic acid residue of formula Q-CH(R)—CO, wherein Q means 1-3 carbon alkoxycarbonylamino group, R means as defined above, and Xbb represents L-proline or L-azetidine-2-carboxylic acid residue. When Xaa, α-substituted alkyl acid, is an α-methylamino or α-hydroxy acid, i.e. Q represents a methylamino or a hydroxyl group, the two residue acid component can be shown as P-D-Xaa-Xbb wherein P represents an N-protecting group such as benzyloxycarbonyl (Z) or tert-butoxycarbonyl (Boc) group or an O-protecting group, preferably tetrahydropyranyl (THP) group.

The acyl dipeptide used as starting material for the α-amino or α-methylamino acid residue-containing compounds is prepared by acylating the x-amino acid with the corresponding chloroformic acid ester to yield 1-3 carbon alkoxycarbonylamino acid and benzyloxycarbonylamino acid, which are then coupled to L-proline or L-azetidine-2-carboxylic acid to yield D-Xaa-Xbb and Z-aminoacyl Xbb that is N-methylated to yield the required P-D-Xaa-Xbb.

D-Xaa, required for the coupling to Xbb, can advantageously be prepared by acetylating the racemic DL-Xaa compound, converting the DL-acetylamino acid to its methyl ester and enzymatically resolving the acetyl-DL-Xaa-OMe racemic ester. The acetyl-D-Xaa-OMe thus obtained is then saponified and deacetylated then converted to the needed N-protected D-amino acid.

The required D-α-hydroxy acid can advantageously be obtained from the corresponding D-α-amino acid. Then it is converted to its O-protected form and coupled to Xbb to yield the needed P-D-Xaa-Xbb.

In a second aspect, the invention provides a pharmaceutical composition comprising a peptidyl arginal according to the first aspect of the invention and a pharmaceutically acceptable carrier, excipient or diluent.

The pharmaceutical formulations comprise an effective amount of a compound of general formula (I) or a pharmaceutically acceptable salt thereof and known pharmaceutically acceptable carriers, filling materials, diluents and/or other pharmaceutical excipients.

The above carriers, diluents or filling materials can be water, alcohols, gelatin, lactose, saccharose, starch, pectin, magnesium stearate, stearic acid, talcum, various oils of animal or plant origin, furthermore glycols, e.g. propylene glycol or polyethylene glycol.

The pharmaceutical excipients can be preservatives, various natural or synthetic emulgeators, dispersing or wetting agents, colouring materials, flavouring agents, buffers, materials promoting disintegration and other materials improving the bioavailability of the active ingredient.

The pharmaceutical compositions of the invention can be prepared in usual formulations such as oral compositions (administered through the mouth such as tablets, capsules, powders, pills, dragées or granulates) as well as parenteral compositions (drugs administered by avoiding the gastrointestinal system such as injections, infusions, suppositories, plasters or ointments).

In a third aspect, the invention provides a method for treating a patient having disseminated intravascular coagulation, the method comprising administering to a patient having disseminated intravascular coagulation a peptidyl arginal corresponding to the formula Xaa-Xbb-Arg-H (I), wherein Xaa and Xbb defined as above, or a pharmaceutically acceptable acid-addition salt thereof. Peptidyl arginals are also referred to as peptidyl arginine aldehyde derivatives.

According to this aspect of the invention, the invention provides a method for treating disseminated intravascular coagulation, the method comprising administering to an animal patient, including a human patient, peptidyl arginals according to the invention. In the method according to this aspect of the invention a therapeutically effective amount of a peptidyl arginal according to the invention is administered for a therapeutically effective period of time to an animal, including a human, which has disseminated vascular coagulation in its body. Preferably, such administration should preferably be intravenous or subcutaneous, most preferably intravenous. Administration of the therapeutic compositions can be carried out using known procedures at dosages and for periods of time effective to reduce symptoms or surrogate markers of DIC. When administered systemically, the therapeutic composition is preferably administered at a sufficient dosage to attain a blood level of peptidyl arginals from about 6 μM to about 100 μM. Preferably, a total dosage will range from about 0.1 mg to about 50 mg peptidyl arginal per kg body weight per day. It may desirable to administer simultaneously, or sequentially a therapeutically effective amount of one or more of the therapeutic compositions of the invention to an individual as a single treatment episode.

The following examples are intended to further illustrate certain particularly preferred embodiments of the invention and are not intended to limit the scope of the invention. Except as otherwise stated, for the following experiments, human thrombin (3,000 NIH U/mg), human albumin and human fibrinogen were obtained from Sigma Aldrich Kft. (Budapest, Hungary) and human factor Xa (8 μg/p from Enzyme Research Laboratories (Swansea, UK). APTT reagent was from REANAL (Budapest Hungary and PT reagent, Simplastin D, was purchased from ORGANON, TEKNIKA (Eppelheim, Germany.

Abbreviations of amino acids, peptides, substituents, and reagents are used in accordance with the IUPAC-IUB conventions. Such abbreviations occurring in this application are as follows. Arg=L-arginine, Boc=tert-butoxy-carbonyl, Bzl=benzyl, Chg=L-cyclohexylglycine, DCHA=dicyclohexylamine, DHP=dihydrpyrane, Eoc=ethoxycarbonyl, Gly=glycine, Me=MePhe=N-methyl-L-phenylalanine, Moc=methoxycarbonyl, Pro=L-proline, pNA=p-nitroanilino, TFA=trifluoroacetic acid, THP=terahydropyranyl, Tos=p-toluenesulfonyl, Z=benzyloxycarbonyl, RT=room temperature. Abbreviations of unusual acids used in this application are Ada=adamantyl-L-alanine, Aze=L-azetidine-2-carboxylic acid, N-Me-D-cHpa=N-methyl-D-cycloheptyalanine, D-cHpa=D-cycloheptylalanine or (R)-2-amino-3-cycloheptylpropionic acid, D-Hla=D-cyclo-heptyllactic acid or (R)-2-hydroxy-3-cycloheptylpropionic acid.

The R_{f} values recorded in the examples were determined by thin-layer chromatography, using silica gel as adsorbent (DC-Alufolien Kieselgel 60 F₂₅₄, Merck Darmstadt), in the following developing systems. The numbers of the systems used are given in brackets after abbreviation R_{f}.
1. Ethyl acetate
2 Ethyl acetate-n-hexane (1:4)
3 Ethyl acetate-n-hexane (1:1)
4 Ethyl acetate-cyclohexane (15:85)
5 Chloroform-acetone (95:5)
6 Ethyl acetate-pyridine-acetic acid-water (960:20:6:11)
7 Ethyl acetate-pyridine-acetic acid-water (480:20:6:11)
8 Ethyl acetate-pyridine-acetic acid-water (240:20:6:11)
9 Ethyl acetate-pyridine-acetic acid-water (120:20:6:11)
10 Ethyl acetate-pyridine-acetic acid-water (90:20:6:11)
11 Ethyl acetate-pyridine-acetic acid-water (60:20:6:11)
12 Ethyl acetate-pyridine-acetic acid-water (45:20:6:11)
13 Ethyl acetate-pyridine-acetic acid-water (30:20:6:11).

The capacity factors (k′) specified in the examples were determined with the apparatus “Pharmacia LKB Analytical HPLCSystem Two” as follows:
Column: LiChrospher RP-18: 12 μm 240×4 mm
Column temperature: ambient
Eluents: Solvent A, 0.1% TFA/water, Solvent B, 0.1% TFA/acetonitrile
Gradient profile: 0→15 min. 30→60% B, then isocratic 60% B.
Solvent flow rate: 1 ml/min.
Detector. LKB 2141 UV Monitor, wavelength: 214 nm.
Injector: Rheodyne 7125. Sample loop: 100 μL.
Pumps: 2 LKB 2148 type. Controlling System: LKB HPLC Manager.
Sample concentration: 1 mg/ml in Solvent A, injected volume 25 μL.
Analysis time 40 min.

The acyl-arginine aldehydes are present in equilibrium structures, i.e. in aldehyde, aldehyde hydrate, and two aminocyclol forms. During HPLC analysis the aldehyde hydrate and one or both aminocyclol forms appear as two or three separate peaks. The acylarginine aldehydes described in the examples are specified by two or three k′ values.

Mass spectrometry. The FAB positive ionization measurements were performed in a Finnigan MAT 8430 apparatus. Samples were dissolved in m-nitrobenzyl alcohol (NBA) matrix and introduced directly into the ion source. In the spectrum of peptidyl-arginine aldehydes an additional molecule ion was detectable, that of the addition compound formed with NBA: [M+H]⁺ and [M+H+NBA]⁺. In the examples the FAB spectra data were specified accordingly. The ESI positive ionization measurements were performed in a VG Quattro (Fisons) apparatus. The samples were dissolved in a mixture of acetonitrile-water (1:1) containing 1% (v/v) of formic acid and were introduced with a 10 ml sample-loop into the ion source at a flow rate of 15-25 ml/min.

### EXAMPLE 1

### Synthesis of ethoxycarbonyl-D-cycloheptylalanyl-L-prolyl-L-arginine Aldehyde Hemisulfate

### Step 1: Ethoxycarbonyl-D-cycloheptylalanyl-L-prolyl-N^{G}-benzyloxycarbonyl-L-arginine Lactam

7.85 g (20.1 mM) of tert-butyloxycarbonyl-N^{G}-benzyloxycarbonyl-L-arginine lactam [(Bajusz et al, J. Med. Chem. 33, 1729 (1990)] was suspended in 20 ml of chloroform, then 20 ml of ethyl acetate saturated with HCl gas (0.11-0.15 g/mA was added with stirring and ice-cooling. The cleaving of the Boc group was monitored by thin-layer chromatography [R_{f} (11)=0.5 (free compound; 1.0 (Boc-compound)]. By the end of the reaction the suspension was diluted with 40 ml of diethyl ether, the crystal mass formed was filtered, washed with 10 ml of acetone and 10 ml of diethyl ether, and dried at reduced pressure over KOH. The resulting N^{G}-benzyloxycarbonyl-L-arginine lactam hydrochloride was dissolved in 20 ml of dimethylformamide, cooled to −20° C. and added to the following mixed anhydride.

7.12 g (20.1 mM) of ethoxycarbonyl-D-cycloheptylalanyl-L-proline (Example 1, Step J) was dissolved in 20 ml of dimethylformamide, cooled to −15° C., then with stirring 2.23 ml (20.1 mM) of N-methyl-morpholine and 2.65 ml (20.1 mM) of isobutyl chloroformate were added. After 10 minutes of stirring the above dimethylformamide solution of N^{G}-benzyloxycarbonyl-L-arginine lactam was added then triethylamine in a quantity to adjust the pH of the reaction mixture to 8 (about 2.8 ml was required). The reaction mixture was stirred at −10° C. for 30 minutes, then at 0° C. for one hour. Thereafter the salts were filtered off and the filtrate was diluted with 100 ml of ethyl acetate. The resulting solution was washed with 3×25 ml of water, 10 ml of 1 M KHSO₄ and 3×10 ml of water, dried over anhydrous Na₂SO₄, and evaporated at 2.0-2.5 kPa. The product obtained was submitted to silica gel column chromatography using 200 g of Kieselgel 60 (0.040-0.063 mm) as adsorbent and ethyl acetate as eluent. The fractions containing solely the pure product [(R_{f} (1)=0.60] were pooled and evaporated at 2.0-2.5 kPa. The evaporation residue was crystallized from diisopropyl ether.

Yield 10.84 g (86,1%), R_{f} (1)=0.55-0.65.

FAB mass spectrum (627 [M+H]⁺) confirmed the assumed structure.

### Step 2: Ethoxycarbonyl-D-cycloheptylalanyl-L-prolyl-N^{G}-benzyloxycarbonyl-L-arginine Aldehyde

8.02 g (12.8 mM) of ethoxycarbonyl-D-cycloheptylalanyl-L-prolyl-N^{G}-benzyloxycarbonyl-L-arginine lactam (Example 1, Step 1) was dissolved in 15 ml of tetrahydrofuran, and then with stirring and at a temperature not exceeding −50° C. a solution of 3.6 mM of LiAlH₄ dissolved in tetrahydrofuran was added. The progress of reduction was monitored by thin-layer chromatography (solvent 7) as developing solvent and, if required, a further portion of LiAlH₄ was added. To this reaction mixture 0.5 M of KHSO₄ was added dropwise with constant stirring and cooling until pH 3 was attained, then 35 ml of water. The resulting solution was extracted with 2×15 ml of hexane, then with 3×20 ml of dichloromethane. The dichloromethane extracts were pooled, washed with 3×15 ml of water, 15 ml of cold 5% sodium hydrogen carbonate solution and again with 15 ml of water, dried over anhydrous Na₂SO₄, and evaporated at 2.0-2.5 kPa. The evaporation residue vas treated with diisopropyl ether, filtered and dried at reduced pressure.

Yield 7.08 g (88%), R_{f} (8)=0.40-0.50.

FAB mass spectrum (629 [M+H]⁺, 782 [M+H+NBA]⁺) confirmed the assumed structure.

### Step 3: Ethoxycarbonyl-D-cycloheptylalanyl-L-prolyl-L-arginine Aldehyde Hemisulfate

6.91 g (11.0 mM) of ethoxycarbonyl-D-cycloheptylalanyl-L-prolyl-N^{G}-benzyloxycarbonyl-L arginine aldehyde (Example 1, Step 2) was dissolved in 85 ml of ethanol and 11.25 ml of 0.5 M of sulfuric acid, then 0.7 g Pd—C catalyst suspended in 14 ml of water was added and the mixture was hydrogenated at about 10° C. The progress of the reaction was monitored by thin-layer chromatography. After completion of the reaction (about 15 minutes), the catalyst was filtered and the filtrate was concentrated to about 7-9 ml at 2.0-2.5 kPa. The residue was diluted with 80 ml of water, extracted with 4×15 ml of dichloromethane and the aqueous solution was left to stand at 20-22° C. for 24 hours. The solution was extracted with 3×15 ml of dichloromethane again and the pH was adjusted to 3.5 with ion-exchange resin Dowex AG 1-X8 (HO⁻), then the solution was freeze-dried.

Yield 4.90 g (82%). R_{f} (11)=0.35-0.45. [α]_{D}²⁰=−77.60 (c=1.018; water).

HPLC: k′=1.695 and 2.328.

FAB mass spectrum (495 [M+H]⁺, 648 [M+H+NBA]⁺) confirmed the assumed structure.

Synthesis of the Starting Materials:

Ethoxycarbonyl-D-cycloheptylalanyl-L-proline

### Step A: 1-cycloheptylacetyl-2,5-dimethylpyrazole

58.6 g (375 mM) cycloheptylacetic acid [Protiva et al, Collect. Czech, Chem., Commun., 55: 1278-1289 (1990)] was dissolved in 375 ml of tetrahydrofuran, cooled to −15° C., then with stifling 41.3 ml (375 mM) of N-methylmorpholine, 49.50 ml (375 mM) of isobutyl chloroformate, and, after 10 minutes of stirring at −10° C. a solution of 37.85 g (393.75 mM) of 3,5-dimethylpyrazole in 300 ml of tetrahydrofuran were added at −10° C. Stirring was continued at −10° C. for 30 min, at 0° C. for an hour, and at room temperature for 3 hours. Thereafter the salts were filtered off, the filtrate evaporated under reduced pressure, and the residue was dissolved in 900 ml of ethyl acetate. The resulting solution was successively washed with 3×80 ml of 1 M NaOH, 89 ml of water 3×80 ml 1 M HCl and with water to neutrality. (Basic washings were combined and acidified to regenerate ˜5.8 g, 37.1 mM, of cycloheptylacetic acid.) The ethyl acetate solution was dried over anhydrous Na₂SO₄, then evaporated at 2.0-2.5 kPa. The resulting oily product was treated as 340 mM of 1-cycloheptylacetyl-2,5-dimethylpyrazole, and used for the next step without further purification. R_{f} (2) 0.8-0.9 (acid: 0.3-0.4).

### Step B: 1-cycloheptylacetaldehyde

To 350 ml of tetrahydrofuran cooled to −30° C. 12.83 g (338 mM) LiAlH₄ was added. Thereafter a solution of the oily product (Example 1, Step A) in 250 ml of cold tetrahydrofuran was introduced dropwise under stirring at −25° C. Reaction was followed by TLC. If required 30-40 ml of a solution of LiAlH₄ in tetrahydrofuran (3 g/100 ml) was added. After completion of the reduction, the cold mixture was acidified with 6 M HCl, and diluted with ethyl acetate (500 ml). The aqueous phase was extracted with ethyl acetate the organic solutions were combined, washed with water to neutrality, dried over anhydrous Na₂SO₄, and then evaporated at 2.0-2.5 kPa. The resulting oily product was crude 1-cycloheptylacetaldehyde contaminated with 2,5-dimethylpyrazole (DMP). R_{f} (2)=0.7-0.8 (DMP: 0.25-0.35).

To the resulting oily product (62.6 g) dissolved in 350 ml of methanol a solution of 36.4 g of NaHSO₃ in 70 ml water was added with stirring. The reaction mixture was kept in refrigerator overnight. The precipitated material was filtered off, washed with a cold mixture of 35 ml methanol and 7 ml of water, then with diethyl ether, and dried. The solid material is the sodium bisulfite adduct (73.87 g, 302.38 mM), R_{f} (14)=0.55-0.65), which was added to a mixture in 450 ml of methylene chloride and 450 ml of water containing 47.7 g (450 mM) of Na₂CO₃. The reaction mixture was stirred overnight. The two phases were separated, the organic phase was washed methylene chloride (2×100 ml). The combined organic layers were washed with water, dried over anhydrous Na₂SO₄, then evaporated at 2.0-2.5 kPa. The resulting oily product (36.97 g, 263.64 mM) was pure 1-cycloheptyl-acetaldehyde, which was directly used for the next reaction. R_{f} (2)=0.7-0.8.

### Step C: 5-cycloheptylmethylhydantoin

To a solution of the aldehyde (Example 1, Step B) in 50% aqueous ethanol (857 ml; 3.25 ml/mM) 15.5 g (290 mM; 1.1 equiv.) ammonium chloride, 27.87 g (659 mM; 2.5 equiv.) ammonium carbonate, and 18.9 g (290 mM 1.1 equiv.) potassium cyanide were added at 50-55° C. with stirring, and stirring was continued at 50° C. for 48 hours. Precipitated material was filtered off, washed with 50% ethanol (100 ml), and dried in a vacuum desiccator. As the first crop, 42.26 g (206.97 mM) material was obtained. Of the mother liquor ethanol was distilled off, the residue was extracted ethyl acetate (1×250 ml and 2×100 ml), the organic solutions were combined, washed with water (3×50 ml), dried over anhydrous Na₂SO₄, then evaporated at 2.0-2.5 kPa. Residue was triturated with diisopropyl ether, filtered and dried. As a second crop, 3.6 g (17.12 mM) material was obtained; total yield 45.86 g (218 mM, 82.5%) of 5-cycloheptymethylhydantoin was obtained. Mp: 240.9° C. R_{f} (6)=0.73-0.78.

Analysis for C₁₁H₁₈N₂O₂ (210.28). Calculated. C %=62.83; H % 8.63; N %=13.32. Found: C % 63.09; H %=8.67; N %=13.25.

### Step D: DL-cycloheptylalanine Hydrochloride

87 g (1.55 M) of KOH was dissolved in 435 ml of n-butanol with stirring and warming, and 45.71 g (217.4 mM) of 5-cycloheptymethylhydantoin (Example 1, Step C) was added. The solution thus obtained was refluxed for 72 hours, then diluted with water and evaporated under reduced pressure. The residue was dissolved in 220 ml of water, acidified to pH 2 with cc HCl (˜130 ml), and kept in refrigerator overnight. The precipitated material was filtered off, washed with 50 ml of water, and dried in vacuum desiccator. The product thus obtained (106.5 g) was taken as 217 mM of DL-cycloheptylalanine, and used for further reactions. R_{f} (11)=0.2-0.3.

### Step E: DL-cycloheptylalanine Methyl Ester Hydrochloride

23.65 ml (325.25 mM) SOCl₂ was dropped into 217 ml of methanol between 0° C. and −5° C. with stirring. Thereafter DL-cycloheptylalanine (217 mM from Example 1, Step D) was added and stirred for 24 hours. Conversion was followed by TLC R_{f} (11)=0.75-0.85 (ester), (0.3-0.4 (acid). When unreacted amino acid could be detected, the reaction mixture was cooled to −5° C., 11.8 ml of SOCl₂ was dropped into, and the reaction mixture was stirred for more 24 hours. Thereafter the undissolved salts were filtered off, washed with methanol (2×50 ml), and the combined methanol solutions were evaporated. The residue was redissolved in methanol and evaporated again. Finally, the residue was triturated with diisopropyl ether, filtered off, washed with diisopropyl ether, and dried in vacuum desiccator over KOH and P₂O₅. 33.68 g (142.85 mM) of DL-cycloheptylalanine methyl ester hydrochloride was obtained. R_{f} (11)=0.5-0.6. Mp. 95.4-96.5° C.

### Step F: Acetyl-DL-cycloheptylalanine Methyl Ester

To a solution of 33.68 g (142.85 mM) of DL-cycloheptylalanine methyl ester hydrochloride (Example 1, Step E) in 140 ml of pyridine, acetic anhydride (16.2 ml, 171.43 m) was added drop-wise during an hour under stirring and cooling in an ice-water bath. Reaction mixture was stirred for 24 hours at ambient temperature then evaporated under reduced pressure. The residue was dissolved in 300 ml ethyl acetate, washed with 1 M KHSO₄ (3×50 ml) and water (3×50 ml), dried over anhydrous Na₂SO₄, and then evaporated at 2.0-2.5 kPa. The resulting oily product was triturated with diisopropyl ether to a solid material that was filtered off, washed with diisopropyl ether then water, and dried in a desiccator. 24.36 g (100.94 mM, 70.4%) of methyl acetyl-DL-cycloheptylalaninate (acetyl-DL-ester) was obtained. R_{f} (1)=0.55-0.65. Mp.: 69-71° C.

Analysis for C₁₃H₂₃NO₃ (241.332). Calculated: C %=64.70; H %=9.61; N %=5.80. Found: C %=64.75; H %=9.76; N % 5.85.

### Step G: Acetyl-D-cycloheptylalanine Methyl Ester (Enzymatic Resolution of acetyl-DL-cycloheptylalanine Methyl Ester)

To a solution of 8.69 g (36 mM) of acetyl-DL-cycloheptylalanine methyl ester, acetyl-DL-eter, (Example 1, Step F) in 36 ml of toluene, 72 ml of water and 36 mg of Subtilisin Carlsberg (Protease Type VIII, Sigma) were added. The enzymatic hydrolysis of the L-enantiomer, acetyl-L-ester, proceeded at pH 7.0, which was maintained by means of an autotitrator, filled with 3 M NaOH. When consumption of NaOH stopped (at 5.8 ml, 17.4 mM) the reaction mixture was diluted with 36 ml of toluene, and the two layers were separated. The aqueous phase was washed with 2×30 ml of toluene. The combined toluene solutions contained the acetyl-D-ester, and the combined aqueous solutions contained the sodium salt of the acetyl-L-acid.

After drying over anhydrous Na₂SO₄, the combined toluene solutions were evaporated under reduced pressure to yield 3.8 g (15.75 mM) of acetyl-D-ester, R_{f} (1) 0.55-0.65, which was directly used in the next step.

The combined aqueous solutions were acidified and extracted with 3×30 ml ethyl acetate. The combined ethyl acetate solutions were washed with water, dried over anhydrous Na₂SO₄, and evaporated under reduced pressure to yield 4.0 g (17.6 mM) of acetyl-L-acid. R_{f} (7)=0.38-0.42.

A similar preparation starting from 9.65 g (40 mM) acetyl-DL-ester (Example 1, Step F) yielded 4.6 g (19.06 mM) acetyl-D-ester and 4.44 g (19.55 mM) acetyl-L-acid.

### Step H D-cycloheptylalanine Hydrochloride

7.24 g (30 mM) of acetyl-D-ester (Example 1, Step G) was suspended in 120 ml 6M HCl and refluxed for 3 hours. The free amino acid was separated as crystals. The reaction mixture was cooled, kept in a refrigerator overnight, filtered, washed with cold water and ether then dried in a vacuum desiccator. 5.9 g (26.69 mM, 89%) of D-cycloheptylalanine hydrochloride was obtained. R_{f} (12)=0.10-0.15. [α]_{D}²⁰=−11° (c=0.4; 1 M HCl).

Analysis for C₁₀H₁₉NO₂.HCl (221.728). Calculated: C %=54.17; H % 9.09; N %=6.32; Cl %=15.99. Found: C % 54.27; H %=9.27; N %=6.30; Cl %=16.2.

### Step I: Ethoxycarbonyl-D-cycloheptylalanine

To a solution of 4.43 g (20 mM) of D-cycloheptylalanine hydrochloride (Example 1, Step 8 in 20 ml of dimethylformamid were added 5.6 ml (40 mM) triethylamine and 3.95 g (21 mM) of (N-hydroxysuccinimidyl)-ethyl carbonate.* After stirring at room temperature for 3 hours the reaction mixture was evaporated, the residue dissolved in 40 ml of ethyl acetate was washed with 2×30 ml 1 M KHSO₄ and with water to neutrality. Thereafter the organic layer was dried over anhydrous Na₂SO₄, then evaporated at 2.0-2.5 kPa. The resulting oily product (4.47 g, ˜17 mg was ethoxycarbonyl-D-cycloheptylalanine [R_{f} (7)=0.85-0.90], which was directly used for in the next step. [α]_{D}²⁰=+6.3° (c=1, methanol).

### *Preparation of N-hydroxysuccinimidyl)-ethyl-carbonate

11.5 g (100 mM) N-hydroxysuccinimide was dissolved in 100 ml of tetrahydrofuran, cooled to −10° C. then under stirring 15.4 ml (110 mM) of triethylamine and 10.45 ml (110 mM) ethyl chlorocarbonate were added. After 2 hours stirring at room temperature, the mixture was filtered, and the filtrate was evaporated under reduced pressure. The oily residue crystallized on cooling. The crystalline material was suspended in light petroleum ether, filtered off, and dried in a vacuum desiccator. Yield 12.78 g (68.3%). Mp.: 39.4-39.7° C.

Analysis for C₇H₉NO₅, (187.15). Calculated: C, 44.92; H, 4.85; N, 7.48. Found: C %=44.67; H %=4.81; N %=7.27.

### Step J: Ethoxycarbonyl-D-cycloheptylalanyl-proline

To a solution of ethoxycarbonyl-D-cycloheptylalanine (˜17 mM, Example 1, Step I) in 17 ml THF was combined with 3.74 g (20 mM) of N-hydroxysuccinimide, cooled to 10° C., and combined with a solution of 4.12 g (20 mole) of 1,3-dicyclohexylcarbodiimide in about 20 ml of THF. The mixture was stirred for about 5 h at 22° C. after which formation of the active ester was judged complete by TLC

L-Proline (1.95 g, 17 mM) was added to the stirred reaction mixture followed by the addition of 2.3 ml (17 mM) of triethylamine. The reaction mixture was stirred at 22° C. for about 15 h after which consumption of the active ester was determined complete by TLC. The reaction mixture was filtered, the filter cake was washed with 10 ml THF, and the filtrate was evaporated. The residue was dissolved in 30 ml of ethyl acetate and 30 ml of water. The aqueous phase was washed with 2×20 ml ethyl acetate, acidified with 20 ml of 1 M KHSO₄, and extracted with 3×20 ml of ethyl acetate. The combined ethyl acetate solutions were washed with water to neutral, dried over anhydrous Na₂SO₄, then evaporated at 2.0-2.5 kPa. On trituration with diisopropyl ether the residue crystallized. This crystal suspension was cooled in a refrigerator, filtered with light petroleum ether, and dried in a vacuum desiccator. 4.2 g (11.85 mM, 70%) ethoxycarbonyl-D-cycloheptylalanyl-L-proline was obtained. R_{f} (7)=0.45-0.55.

Analysis for C₁₈H₃₀N₂O₄ (354.45). Calculated: C, 60.99; H, 8.53; N, 7.90. Found: C %=60.14; H %=8.55; N %=7.38.

FAB mass spectrum (355 [M+H]⁺) confirmed the assumed structure.

### EXAMPLE 2

### Synthesis of N-methyl-D-cycloheptylalanyl-L-prolyl-L-arginine Aldehyde Sulfate

### Step 1: Benzyloxycarbonyl-N-methyl-D-cycloheptylalanyl-L-prolyl-N^{G}-benzyl-oxycarbonyl-L-arginine Lactam

3.93 g (10 mM) of tert-butyloxycarbonyl-N^{G}-benzyloxycarbonyl-L-arginine lactam [(Bajusz et al, J. Med. Chem. 33, 1729 (1990)] was suspended in 10 ml of chloroform, then 10 ml of ethyl acetate saturated with HCl gas (0.11-0.15 g/ml) was added with stirring and ice-cooling. The cleaving of the Boc group was monitored by thin-layer chromatography [R_{f} (11) 0.5 (free compound); 1.0 (Boc-compound)]. By the end of the reaction the suspension was diluted with 20 ml of diethyl ether, the crystal mass formed was filtered, washed with 5 ml of acetone and 5 ml of diethyl ether, and dried at reduced pressure over KOH. The resulting N^{G}-benzyloxycarbonyl-L-arginine lactam hydrochloride was dissolved in 10 ml of dimethylformamide, cooled to −20° C. and added to the following mixed anhydride.

4.32 g (10 ml) of benzyloxycarbonyl-N-methyl-D-cycloheptylalanyl-L-proline (Example 2, Step C) was dissolved in 10 ml of dimethylformamide, cooled to −15° C., then with stirring 1.12 ml (10.1 mM) of N-methyl-morpholine and 1.33 ml (10.1 mM) of isobutyl chloroformate were added. After 10 minutes of stirring the above dimethylformamide solution of N^{G}-benzyloxycarbonyl-L-arginine lactam was added then triethylamine in a quantity to adjust the pH of the reaction mixture to 8 (about 1.4 ml was required). The reaction mixture was stirred at −10° C. for 30 minutes, then at 0° C. for one hour. Thereafter the salts were filtered off and the filtrate was diluted with 100 ml of ethyl acetate. The resulting solution was washed with 3×15 ml of water, 6 ml of 1 M KHSO₄ and 3×6 ml of water, dried over anhydrous Na₂SO₄, and evaporated at 2.0-2.5 kPa. The product obtained was submitted to silica gel column chromatography using 100 g of Kieselgel 60 (0.040-0.063 mm) as adsorbent and ethyl acetate as eluent. The fractions containing solely the pure product [(R_{f} (1)=0.70] were pooled and evaporated at 2.0-2.5 kPa. The evaporation residue was crystallized from diisopropyl ether.

Yield 6.0 g (85%), R_{f} (1)=0.65-0.75.

FAB mass spectrum (703 [M+H]⁺) confirmed the assumed structure.

### Step 2: Benzyloxycarbonyl-N-methyl-D-cycloheptylalanyl-L-prolyl-N^{G}-benzyloxycarbonyl-L-arginine Aldehyde

5.62 g (8 mM) of benzyloxycarbonyl-N-methyl-D-cycloheptylalanyl-L-prolyl-N^{G}-benzyloxycarbonyl-L-arginine lactam (Example 2, Step 1) was dissolved in 10 ml of tetrahydrofuran, and then with stirring and at a temperature not exceeding −50° C. a solution of 2.25 mM of LiAlH₄ dissolved in tetrahydrofuran was added. The progress of reduction was monitored by thin-layer chromatography (solvent 7) as developing solvent and, if required, a further portion of LiAlH₄ was added. To this reaction mixture 0.5 M of KHSO₄ was added drop-wise with constant stirring and cooling until pH 3 was attained, then 25 ml of water. The resulting solution was extracted with 2×10 ml of hexane, then with 3×15 ml of dichloromethane. The dichloromethane extracts were pooled, washed with 3×15 ml of water, 15 ml of cold 5% NaHCO₃ solution and again with 15 ml of water, dried over anhydrous Na₂SO₄, and evaporated at 2.0-2.5 kPa. The evaporation residue was treated with diisopropyl ether, filtered and dried at reduced pressure.

Yield 4.95 g (88%), R_{f} (1)=0.20-0.25.

FAB mass spectrum (705 [M+H]⁺, 858 [M+H+NBA]⁺) confirmed the assumed structure.

### Step 3: N-methyl-D-cycloheptylalanyl-L-prolyl-L-arginine Aldehyde Sulfate

4.6 g (6.5 mM) of benzyloxycarbonyl-N-methyl-D-cycloheptylalanyl-L-prolyl-N^{G}-benzyloxycarbonyl-L-arginine aldehyde (Example 2, Step 2) was dissolved in 65 ml of ethanol and 13.5 ml of 0.5 M of sulfuric acid, then 0.4 g Pd—C catalyst suspended in 10 ml of water was added and the mixture was hydrogenated at about 10° C. The progress of the reaction was monitored by thin-layer chromatography. After completion of the reaction (about 15 minutes), the catalyst was filtered and the filtrate was concentrated to about 5-7 ml at 2.0-2.5 kPa. The residue was diluted with 50 ml of water, extracted with 4×10 ml of dichloromethane and the aqueous solution was left to stand at 20-22° C. for 24 hours. The solution was extracted with 3×10 ml of dichloromethane again and the pH was adjusted to 3.5 with ion-exchange resin Dowex AG 1-X8 (HO⁻), then the solution was freeze-dried.

Yield 2.85 g (82%). R_{f} (9)=0.35-0.45. [α]_{D}²⁰=−79.6° (c=1; water).

FAB mass spectrum (437 [M+H]⁺, 590 [M+H+NBA]⁺) confirmed the assumed structure.

Synthesis of the Starting Materials:

Benzyloxycarbonyl-N-methyl-D-cycloheptylalanyl-L-proline

### Step A. Synthesis of N-benzyloxycarbonyl-D-cycloheptylalanine

D-Cycloheptylalanine hydrochloride (Example 1, Step H) (11.09 g, 50 mM) was combined with 40 ml of THF and 40 ml of water at 0° C. The stirred mixture was adjusted to about pH 10 by the addition of 5M NaOH solution. Benzyl chloroformate (8.22 ml, 55.4 mM) was added to the reaction mixture while maintaining a temperature of about 3° C. and approximately pH 10 by the addition of 5M NaOH as required. Upon completion of the benzyl chloroformate addition, the reaction mixture was stirred for 1 h at 0° C. and maintained at about pH 10. Stirring was continued overnight at RT and completion of the reaction was checked by TLC (7). If required, a further quantity of benzyl chloroformate (1-2×0.75 ml, 5 mM) was added to the reaction mixture while maintaining a temperature of about 3° C. and approximately pH 10 by the addition of 5M NaOH t-Butyl methyl ether (25 ml) was added, and the stirred mixture was warmed to 22° C. The aqueous phase was separated and washed with a second 25 ml portion of t-butyl methyl ether. Content of the organic phase was checked by TLC and, if required, the organic phase was back-extracted with 25 ml water. The aqueous phases and 25 ml of ethyl acetate were combined and adjusted to pH 2 with concentrated HCl. The phases were separated, and the aqueous phase was extracted with a second 25 ml portion of ethyl acetate. The combined organic phase was washed with 20 ml of 1 M KHSO₄ and 2×30 ml water, dried over anhydrous Na₂SO₄, and evaporated at 2.0-2.5 kPa. The evaporation residue was dissolved in 45 ml THF, and this solution of benzyloxycarbonyl-D-cycloheptylalanine was held for use in the next step without further purification.

### Step B: Benzyloxycarbonyl-D-cycloheptylalanyl-proline

TFA solution of benzyloxycarbonyl-D-cycloheptylalanine obtained in Example 2, Step A was combined with 5.9 g (51.24 ml) of N-hydroxysuccinimide, cooled to 10° C., and combined with a solution of 11 g (53.3 mM) of 1,3-dicyclohexylcarbodi-imide in about 25 ml of THF. The mixture was stirred for about 4.5 h at 22° C. after which formation of the active ester was judged complete by TLC.

L-Proline (5.9 g, 51.24 mM) was added to the stirred reaction mixture followed by the addition of 7.2 ml (51.24 mM) of triethylamine. The reaction mixture was stirred at 22° C. for about 15 h after which consumption of the active ester was determined complete by TLC. The reaction mixture was filtered, the filter cake was washed with 25 ml THE, and the filtrate was evaporated. The residue was dissolved in 50 ml of ethyl acetate and 50 ml of water. The aqueous phase was washed with 2×20 ml ethyl acetate, acidified with 20 ml of 1 M KHSO₄, and extracted with 3×40 ml of ethyl acetate. The combined ethyl acetate solutions were washed with water to neutral, dried over anhydrous Na₂SO₄, then evaporated at 2.0-2.5 kPa. The residue, benzyloxycarbonyl-D-cycloheptylalanyl-L-proline, was dissolved in 60 ml of THF, and was used in the next step without further purification.

### Step C: Benzyloxycarbonyl-N-methyl-D-cycloheptylalanyl-L-proline

Iodomethane (17.95 ml, 288 mM) was added to the THF solution of benzyloxycarbonyl-D-cycloheptylalanyl-L-proline from Example 2, Step B. This solution was cooled to 8° C. and transferred along with a 20 ml THF rinse to a stirred slurry of 4.75 g (119 mM) of sodium hydride 60% in 35 ml of THF while maintaining the temperature below 13° C. The reaction mixture was stirred at 11° C. for 24 h. Excess sodium hydride was decomposed by the careful addition of 1.6 ml of water to the reaction while maintaining the temperature below 13° C. and controlling foaming. The quenched reaction mixture was stirred for about 20 min at 22° C. and then concentrated to about 40 ml under reduced pressure at a temperature below 30° C. Water (70 ml) was added to the residue followed by 30 ml of t-butyl methyl ether. The phases were separated, and the aqueous phase was washed again with 30 ml of t-butyl methyl ether. The aqueous product phase was combined with 40 ml of ethyl acetate and adjusted to pH 2.2 with 3M sulfuric acid solution. The phases were separated, and the aqueous phase was back-extracted with 40 ml of ethyl acetate. The combined organic phase was washed with 70 ml of a 5% sodium thiosulfate solution. The phases were separated, and the organic phase was concentrated to a small volume under reduced pressure vacuum (33-45 kPa) while maintaining the temperature below 50° C. The residue was combined with 18.6 ml of THF and 100 ml of water and adjusted to pH 8.5 with cyclohexylamine. The resulting slurry was concentrated to 100 ml under reduced pressure (9-33 kPa) at 25 to 55° C., adjusted to 25° C., diluted with 71.5 ml of water and stirred for about 10.5 h. The slurry was filtered, washed with water and air dried at 45° C. to afford 16.72 g of benzyloxycarbonyl-N-methyl-D-cycloheptylalanyl-L-proline cyclohexylamine salt (63% yield from D-cycloheptylalanine). R_{f} (8)=0.55-0.65.

### EXAMPLE 3

### Synthesis of D-cycloheptyllactyl-L-prolyl-L-arginine Aldehyde Hemisulfate

### Step 1: Tetrahydropyranyl-D-cycloheptyllactyl-L-prolyl-N^{G}-benzyloxycarbonyl-L-arginine Lactam

5.08 g (13 mM) of tert-butyloxycarbonyl-N^{G}-benzyloxycarbonyl-L-arginine lactam [(Bajusz et al, J. Med. Chem. 33, 1729 (1990)] was suspended in 13 ml of chloroform, then 13 ml of ethyl acetate saturated with HCl gas (0.11-0.15 g/ml) was added with stirring and ice-cooling. The cleaving of the Boc group was monitored by thin-layer chromatography [R_{f} (11)=0.5 (free compound); 1.0 (Boc-compound)]. By the end of the reaction the suspension was diluted with 25 ml of diethyl ether, the crystal mass formed was filtered, washed with 7 ml of acetone and 7 ml of diethyl ether, and dried at reduced pressure over potassium hydroxide. The resulting N^{G}-benzyloxycarbonyl-L-arginine lactam hydrochloride was dissolved in 13 ml of dimethylformamide, cooled to −20° C. and added to the following mixed anhydride.

The solution of tetrahydropyranyl-D-cycloheptyllactyl-L-proline triethyl-ammonium salt obtained in Step I of Example 3 (12 mM) was cooled to −20° C. then with stirring 1.6 ml (12 mM) of isobutyl chloroformate was added. After 10 minutes of stirring the above dimethylformamide solution of N^{G}-benzyloxycarbonyl-L-arginine lactam was added then triethylamine in a quantity to adjust the pH of the reaction mixture to 8 (about 1.8 ml was required). The reaction mixture was stirred at −10° C. for 30 minutes, then at 0° C. for one hour. Thereafter the salts were filtered off and the filtrate was diluted with 65 ml of ethyl acetate. The resulting solution was washed with 3×25 ml of water, 7 ml of 1 M potassium hydrogen sulfate and 3×7 ml of water, dried over anhydrous Na₂SO₄ and evaporated at 2.0-2.5 kPa. The product obtained was submitted to silica gel column chromatography using 130 g of Kieselgel 60 (0.040-0.063 mm) as adsorbent and ethyl acetate as eluent. The fractions containing solely the pure product [(R_{f} (1)=0.60] were pooled and evaporated at 2.0-2.5 kPa. The evaporation residue was crystallized from diisopropyl ether.

Yield 5.0 g (7.8 mM, 64%), R_{f} (1)=0.6

FAB mass spectrum (640 [M+H⁺) confirmed the assumed structure.

### Step 2: Tetrahydropyranyl-D-cycloheptyllactyl-L-prolyl-N^{G}-benzyloxycarbonyl-L-arginine Aldehyde

4.8 g (7.51 mM) of tetrahydropyranyl-D-cycloheptyllactyl-L-N^{G}-benzyloxycarbonyl-L-arginine lactam (Example 3, Step 1) was dissolved in 15 ml of tetrahydrofuran, then with stirring at a temperature not exceeding −50° C. a solution of 3.6 mM of lithium aluminum hydride dissolved in tetrahydrofuran was added. The progress of reduction was monitored by thin-layer chromatography with developing solvent no. 8 and, if required, a further portion of lithium aluminum hydride was added. To this reaction mixture 0.5 M of sulfuric acid was added dropwise with constant stirring and cooling until pH 3 was attained, then 35 ml of water is added. The resulting solution was extracted with 2×15 ml of hexane, then with 3×20 ml of dichloromethane. The dichloromethane extracts were pooled, washed with 3×15 ml of water, 15 ml of cold 5% sodium hydrogen carbonate solution and again with 15 ml of water, dried over anhydrous sodium sulfate and evaporated at 2.0-2.5 kPa. The evaporation residue was treated with diisopropyl ether, filtered and dried at reduced pressure.

Yield 3.9 g (6.07 mM, 81%). R_{f} (8)=0.40. [α]_{D}²⁰=+16.0° (c=1, tetrahydrofuran).

FAB mass spectrum (642 [M+H⁺, 795 [M+H+NBA]⁺) confirmed the assumed structure.

### Step 3: D-Cycloheptyllactyl-prolyl-L-arginine Aldehyde Hemisulfate

3.21 g (5 mM) of tetrahydropyranyl-D-cycloheptyllactyl-L-prolyl-N^{G}-benzyloxycarbonyl-L-arginine aldehyde (Example 3, Step 2) was dissolved in 40 ml of ethanol and 5 ml of 0.5 M of sulfuric acid, then 0.3 g Pd—C catalyst suspended in 6 ml water was added and the mixture was hydrogenated at about 10° C. The progress of the reaction was monitored by thin-layer chromatography. After completion of the reaction (about 15 minutes), the catalyst was filtered off and the filtrate was concentrated to about 4-6 ml at 2.0-2.5 kPa. The residue was diluted with 40 ml of water, extracted with 4×7 ml of dichloromethane and the aqueous solution was left to stand at 20-22° C. for 24 hours. The solution was extracted with 3×15 ml of dichloromethane again and the pH was adjusted to 3.5 with ion-exchange resin Dowex AG 1-X8 (HO⁻), then the solution was freeze-dried.

Yield 1.65 g (3.5 mM, 70%) [α]_{D}²⁰=−94.7° (c=1, water)

HPLC: k′=2.702 and 3.010.

FAB mass spectrum (424 [M+H]⁺, 577 [M+H+NBA]⁺) confirmed the assumed structure.

Synthesis of the Starting Materials:

Tetrahydropyranyl-D-cycloheptyllactyl-L-proline triethylammonium salt

### Step A: Chloroacetyl-DL-cycloheptylalanine Methyl Ester

To a solution of 15.2 g (65 mM) of DL-cycloheptylalanine methyl ester hydrochloride (Example 1, Step E) in 65 ml of dichloromethane 9.1 ml (65 mM) triethylamine and 14.9 g (78 mM) of (N-hydroxysuccinimidyl)-chloroacetate* were added. After stirring at room temperature for 3 hours, the reaction mixture was diluted with 65 ml of dichloromethane and successively washed with 3×30 ml of water, 1 M KHSO₄, water, 5% NaHCO₃, and finally with water to neutrality. Thereafter the organic layer was dried over anhydrous Na₂SO₄, then evaporated at 2.0-2.5 kPa. The resulting oily product was triturated with light petroleum ether. The solid material was filtered off, washed with light petroleum ether, and dried in a vacuum desiccator. 17.54 g (63.6 mM, ˜98%) of chloroacetyl-DL-cycloheptylalanine methyl ester was obtained, which was directly used for the next reaction. R_{f} (7)=0.73-0.83. Mp.: 78-80° C.

Analysis for C₁₃H₂₂NO₃Cl (275.777). Calculated. C %=56.62; H %=8.04; N % 5.08; Cl %=12.86. Found: C %=55.65; H %=7.93; N %=5.06; Cl %=12.72.

*Preparation of (N-hydroxysuccinimidyl) Chloroacetate

32 ml (450 mM) of chloroacetyl chloride was added to 23 g (200 mM) of N-hydroxysuccinimide and the mixture was refluxed for 10 minutes then poured onto crushed ice, filtered, washed with cold water, and dried in a vacuum desiccator. Yield 20.23 g (105.9 mM, 53%). Mp.: 113.3-113.7° C.

Analysis for C₅H₆NO₄Cl C₇H₉NO₅ (191.55). Calculated: C %=37.62; H %=3.16; N %=7.31; Cl % 18.51. Found C %=37.37; H % 3.16; N % 7.23; Cl %=18.35.

### Step B: Chloroacetyl-D-cycloheptylalanine Methyl Ester (Enzymatic Resolution of chloroacetyl-DL-cycloheptylalanine Methyl Ester)

To a solution of 8.71 g (31.6 mole) of chloroacetyl-DL-cycloheptylalanine methyl ester, DL-ester, (Example 3, Step A) in 30 ml of toluene 70 ml of water and 50 mg of Subtilisin Carlsberg (Protease Type VIII, Sigma) were added. The enzymatic hydrolysis of the L-enantiomer, L-ester, proceeded at pH 7.0, which was maintained by means of an autotitrator, filled with 3 M NaOH. When consumption of NaOH stopped (at 5.522 ml, 16.57 mM), the reaction mixture was diluted with 30 ml of toluene, and the two layers were separated. The aqueous phase was washed with 2×20 ml of toluene. The combined toluene solutions contained the D-ester, and the combined aqueous solutions contained the sodium salt of the L-acid.

After drying over anhydrous Na₂SO₄, the combined toluene solutions were evaporated under reduced pressure to yield 4.18 g (15.16 mM) of D-ester. R_{f} (7)=0.73-0.83, which was directly used for the preparation of D-cycloheptylalanine.

The combined aqueous solutions were acidified and extracted with 3×30 ml ethyl acetate. The combined ethyl acetate solutions were washed with water, dried over anhydrous Na₂SO₄, and evaporated under reduced pressure to yield 3.46 g (13.22 n of L-acid [R_{f} (7)=0.45-0.50], which was directly used for the preparation of L-cycloheptylalanine.

A similar preparation starting from 8.25 g (30 mM) DL-ester (Example 2, Step A) yielded 4.08 g (14.79 mM) D-ester and 3.23 g (12.34 mM) L-acid.

### Step C; D-cycloheptylalanine Hydrochloride

8.27 g (30 no of D-ester (Example 3, Step B) was suspended in 120 ml 6M HCl and refluxed for 3 hours. The free amino acid was separated as crystals. The reaction mixture was cooled, kept in a refrigerator overnight, filtered, washed with cold water and ether, then dried in a vacuum desiccator. 5.9 g (26.69 mM, 89%) of D-cycloheptylalanine hydrochloride was obtained. R_{f} (12)=0.10-0.15. [α]_{D}²⁰=−11° (c=0.4; 1 M HCl.

Analysis for C₁₀H₁₉NO₂.HCl (221.728). Calculated: C % 54.17; H %=9.09; N % 6.32; Cl %=15.99. Found C %=54.27; H %=9.27; N %=6.30; Cl %=16.2.

### Step D: D-cycloheptyllactic Acid Dicyclohexylammonium Salt

5.78 g (26.15 mM) of D-cycloheptylalanine hydrochloride (Example 3, Step C) was dissolved in 26 ml of water, diluted with 105 ml of water and 52.5 ml of glacial acetic acid, and cooled to 5° C. To this inure was dropped a solution of 18.0 g (261 mM) of NaNO₂ in 30 ml of water with stirring and cooling. Stirring was continued at 5° C. for an hour and at room temperature overnight. Next day the reaction mixture was acidified with 25 ml of cc HCl with stirring. Thereafter the mix was evaporated to dryness at 50° C. under reduced pressure. The residue dissolved in 100 ml of water and evaporated similarly, triturated with toluene, and evaporated again. The final residue was dissolved 50 ml of ethyl acetate and 50 ml of water. The aqueous phase was washed with ethyl acetate, and the combined ethyl acetate solutions were washed with water to neutrality, dried over anhydrous Na₂SO₄, and evaporated under reduced pressure. The ensuing solid was dissolved in 20 ml of diisopropyl ether. To this solution, 5 ml (25 mM) of dicyclohexylamine was added, upon which the crystalline salt was separated. After cooling crystals were filtered off, washed with cold ether and dried in a vacuum desiccator to yield 5.5 g (14.96 mM, 57.2%) of D-cycloheptyllactic acid dicyclohexylamine salt, D-cHla.DCHA. R_{f} (7)=0.53-0.60. [α]_{D}²⁰=+19.5° (c=1; methanol.). Mp.: 147-150° C.

Analysis for C₁₀H₁₈O₃ (C₂₂H₄₁NO₃). Calculated: C %=71.89; H % 11.24; N %=3.81. Found: C % 71.57; H %=11.34; N %=3.83.

Conversion of 0.35 g (1 mM) of D-cHla.DCHA into the free α-hydroxy acid yielded 0.15 g (0.8 mM) of D-cHla, [α]_{D}²⁰=+10.1° (c=1; methanol.); mp.: 125-127° C.

Analysis for C₁₀H₁₈O₃ (186.252). Calculated: C %=64.48; H %=9.74. Found. C %=64.54; H %=9.86.

### Step E: D-cycloheptyllactic Acid Benzyl Ester

To a solution of 11.21 g (30.5 mM) D-cycloheptyllactic acid dicyclohexylammonium salt (Example 3. Step D) in 30 ml dimethylformamide 3.57 ml (30 mM) benzyl bromide was added. The mixture was stirred at room temperature for 24 hours then filtered and evaporated at 2.0-2.5 kPa. The residue was dissolved in 20 ml 0.5 M potassium hydrogen carbonate and 60 ml of diethyl ether. The organic phase was successively washed with 20 ml of water, 0.5 M KHSO₄, and water then dried over anhydrous sodium sulfate and evaporated under reduced pressure. The oily residue was 8.3 g (˜30 mM) of D-cycloheptyllactic acid benzyl ester [R_{f} (3)=0.2-0.3], which was directly used in Step F.

### Step F: Tetrahydropyranyl-D-cycloheptyllactic Acid Benzyl Ester

To a solution of 8.29 g (30 mM) D-cycloheptyllactic acid benzyl ester (Example 3, Step E) in 30 ml dichloromethane 3.01 ml (33 mM) 3,4-dihydro-2H-pyran and 0.3 ml ˜3 M HCl in ethyl acetate were added, and the mixture was left to stand at room temperature for 16 hours. Thereafter the reaction mixture was diluted with 40 ml of dichloromethane, washed with 3×20 ml of water, and dried over anhydrous sodium sulfate and evaporated at 2.0-25 kPa. The residue was submitted to silica gel column chromatography using 200 g of Kieselgel 60 (0.040-0.063 mM) as adsorbent and a 85:15 mixture of cyclohexane and ethyl acetate as eluent. The fractions containing solely the pure product [R_{f} (4)=0.60-0.70] were pooled and evaporated at 2.0-2.5 kPa. The oily residue was 7.9 g (21.9 mM, 73%) tetrahydropyranyl-D-cycloheptyllactic acid benzyl ester, which was directly used in the next step.

### Step G: Tetrahydropyranyl-D-cycloheptyllactic Acid Triethylammonium Salt

7.21 g (20 mM) tetrahydropyranyl-D-cycloheptyllactic acid benzyl ester (Example 3, Step F) was dissolved in 20 ml of dimethylformamide, 2.8 ml (20 mM) triethylamine was added and hydrogenated in the presence of 0.1 g of Pd/C catalyst. The progress of the reaction was monitored by thin-layer chromatography [R_{f} (1)=0.30 (ester), 0.00 (acid)]. After completion of the reaction, the catalyst was filtered off and washed with 2×5 ml of dimethylformamide. The filtrate and washings were combined and used in the next step as a solution of 20 mM of tetrahydropyranyl-D-cycloheptyllactic acid triethylammonium salt.

### Step H: Tetrahydropyranyl-D-cycloheptyllactyl-proline Benzyl Ester

The solution of tetrahydropyranyl-D-cycloheptyllactic acid triethylammonium salt obtained in Step G of Example 3 (20 mM) was cooled to +5° C., and with stirring 2.7 g (20 mM) 1-hydroxybenzotriazol, 4.83 g (20 mM) L-proline benzyl ester hydrochloride, and 4.12 g (20 mM) dicyclohexylcarbodiimide were added. The reaction mixture was kept at room temperature overnight then filtered and evaporated at 2.0-2.5 kPa. The residue was dissolved in 50 ml ethyl acetate and washed with 20 ml water and 5% sodium hydrogen carbonate, dried over anhydrous Na₂SO₄ and evaporated under reduced pressure. The residue was submitted to silica gel column chromatography using 200 g of Kieselgel 60 (0.040-0.063 mm) as adsorbent and a 1:1 mixture of n-hexane and ethyl acetate as eluent. The fractions containing solely the pure product [R_{f} (3)=0.3-0.4] were pooled and evaporated at 2.0-2.5 kPa. The oily residue was 5.95 g (13 mM, 65%) tetrahydropyranyl-D-cycloheptyllactyl-L-proline benzyl ester, which was directly used in the next step.

### Step I: Tetrahydropyranyl-D-cycloheptyllactyl-L-proline Triethylammonium Salt

5.5 g (12 mM) of tetrahydropyranyl-D-cycloheptyllactyl-L-proline benzyl ester (Example 3, Step H) was dissolved in 12 ml of dimethylformamide, 1.68 ml (12 mM) triethylamine was added and hydrogenated in the presence of 0.1 g of Pd/C catalyst. The progress of the reaction was monitored by thin-layer chromatography [R_{f} (1)=0.30 (ester), 0.00 (acid)]. After completion of the reaction, the catalyst was filtered off and washed with 2×2 ml of dimethylformamide. The filtrate and washings were combined and used as a solution containing 12 mM of tetrahydropyranyl-D-cycloheptyllactyl-L-proline triethylammonium salt.

### EXAMPLE 4

### Synthesis of N-methyl-D-cyclohexylglycyl-L-azetidine-2-carbonyl-L-arginine Aldehyde Hemisulfate

### Step 1: Benzyloxycarbonyl-N-methyl-D-cyclohexylglycyl-L-azetidine-2-carbonyl-N^{G}-benzyloxycarbonyl-L-arginine Lactam

1.97 g (5 mM) of tert-butyloxycarbonyl-N^{G}-benzyloxycarbonyl-L-arginine lactam [(Bajusz et al, J. Med. Chem. 33, 1729 (1990)] was suspended in 5 ml of chloroform, then 5 ml of ethyl acetate saturated with HCl gas (0.11-0.15 g/ml) was added with stirring and ice-cooling. The cleaving of the Boc group was monitored by thin-layer chromatography [R_{f} (11)=0.5 (free compound); 1.0 (Boc-compound)]. By the end of the reaction the suspension was diluted with 10 ml of diethyl ether, the crystal mass formed was filtered, washed with 3 ml of acetone and 3 ml of diethyl ether, and dried at reduced pressure over KOH. The resulting N^{G}-benzyloxycarbonyl-L-arginine lactam hydrochloride was dissolved in 5 ml of dimethylformamide, cooled to −20° C. and added to the following mixed anhydride.

1.95 g (5 mM) of benzyloxycarbonyl-N-methyl-D-cyclohexylglycyl-L-azetidine-2-carboxylic acid (Example 4, Step B) was dissolved in 5 ml of dimethylformamide, cooled to −15° C., then with stirring 0.56 ml (5.05 mM) of N-methyl-morpholine and 0.665 ml (5.05 mM) of isobutyl chloroformate were added. After 10 minutes of stirring the above dimethylformamide solution of N^{G}-benzyloxycarbonyl-L-arginine lactam was added then triethylamine in a quantity to adjust the pH of the reaction mixture to 8 (about 0.7 ml was required). The reaction mixture was stirred at −10° C. for 30 minutes, then at 0° C. for one hour. Thereafter the salts were filtered off and the filtrate was diluted with 50 ml of ethyl acetate. The resulting solution was washed with 3×7 ml of water, 3 ml of 1 M KHSO₄ and 3×3 ml of water, dried over anhydrous Na₂SO₄, and evaporated at 2.0-2.5 kPa. The product obtained was submitted to silica gel column chromatography using 50 g of Kieselgel 60 (0.040-0.063 mm) as adsorbent and ethyl acetate as eluent. The fractions containing solely the pure product [R_{f} (1)=0.70] were pooled and evaporated at 2.0-2.5 kPa. The evaporation residue was crystallized from diisopropyl ether.

Yield 2.35 g (71%), R_{f} (1)=0.45-0.55

FAB mass spectrum (661 [M+H]⁺) confirmed the assumed structure.

### Step 2: Benzyloxycarbonyl-N-methyl-D-cyclohexyglycyl-azetidine-2-carbonyl-N^{G}-benzyloxycarbonyl-L-arginine Aldehyde

2.15 g (3.25 mM) of benzyloxycarbonyl-N-methyl-D-cyclohexylglycyl-L-azetidine-2-carbonyl-N^{G}-benzyloxycarbonyl-L-arginine lactam (Example 4, Step 1) was dissolved in 5 ml of tetrahydrofuran, and then with stirring and at a temperature not exceeding −50° C. a solution of 2.25 mM of LiAlH₄ dissolved in tetrahydrofuran was added. The progress of reduction was monitored by thin-layer chromatography (solvent 7) as developing solvent and, if required, a further portion of LiAlH₄ was added. To this reaction mixture 0.5 M of KHSO₄ was added drop-wise with constant stirring and cooling until pH 3 was attained, then 13 ml of water. The resulting solution was extracted with 2×5 ml of hexane, then with 3×7 ml of dichloromethane. The dichloromethane extracts were pooled, washed with 3×7 ml of water, 7 ml of cold 5% NaHCO₃ solution and again with 7 ml of water, dried over anhydrous Na₂SO₄, and evaporated at 2.0-2.5 kPa. The evaporation residue was treated with diisopropyl ether, filtered and dried at reduced pressure.

Yield 1.6 g (74%), R_{f} (7)=0.33-0.43

FAB mass spectrum (663 [M+H]⁺) confirmed the assumed structure.

### Step 3: N-methyl-D-cyclohexylglycyl-L-azetidine-2-carbonyl-L-arginine Aldehyde Sulfate

1.53 g (2.3 mM) of benzyloxycarbonyl-N-methyl-D-cyclohexylglycyl-azetidine-2-carbonyl-N^{G}-benzyloxycarbonyl-L-arginine aldehyde (Example 4, Step 2) was dissolved in 23 ml of ethanol and 4.8 ml of 0.5 M of sulfuric acid, then 0.15 g Pd—C catalyst suspended in 3.5 ml of water was added and the mixture was hydrogenated at about 10° C. The progress of the reaction was monitored by thin-layer chromatography. After completion of the reaction (about 15 minutes), the catalyst was filtered and the filtrate was concentrated to about 2-3 ml at 2.0-2.5 kPa. The residue was diluted with 20 ml of water, extracted with 4×4 ml of dichloromethane and the aqueous solution was left to stand at 20-22° C. for 24 hours. The solution was extracted with 3×4 ml of dichloromethane again and the pH was adjusted to 3.5 with ion-exchange resin Dowex AG 1-X8 (HO⁻), then the solution was freeze-dried.

Yield 1.02 g (90%). R_{f} (12)=0.40.

FAB mass spectrum (395 [M+H]⁺, 548 [M+H+NBA]⁺) confirmed the assumed structure.

Synthesis of the Starting Materials:

Benzyloxycarbonyl-N-methyl-D-cyclohexylglycyl-L-azetidine-2-carboxylic acid

### Step A. Synthesis of benzyloxycarbonyl-D-cyclohexylglycine 2,4,5-trichlorophenyl Ester

To a stirred suspension of 5.42 g (20 mM) of D-cyclohexylglycine trifluoracetate salt and 5.6 ml (40 mM) triethylamine in 50 ml dimethylformamide, 8.8 g (22 mM) benzyl-pentachlorophenyl carbonate [Anteunis et al.: Bul. Soc. Chim Belg. 96, 775 (1987)] and 6.16 ml (44 mM) triethylamine were added. After 3 hours stirring the reaction mixture was evaporated under reduced pressure, the residue was dissolved in 60 ml of diethyl ether and 60 of water. The phases were separated, the organic phase was washed with water and the combined aqueous phases were washed with diethyl ether, acidified with 1 M KHSO₄ to pH 3 then extracted with 3×30 ml ethyl acetate. The organic phase was washed with water to neutral, dried over anhydrous Na₂SO₄, and evaporated at 2.0-2.5 kPa.

The evaporation residue is benzyloxycarbonyl-D-cyclohexylglycine that was dissolved in 20 ml tetrahydrofurane and combined 4.54 g (22 mM) 2,4,5-trichloro-phenol and 4.54 g (22 mM) dicyclohexylcarbodiimide. Three hours later the reaction mixture was filtered, the filtrate and washings combined and evaporated under reduced pressure. The solid residue was purified by silica gel column chromatography using 140 g of Kieselgel 60 (0.040-0.063 mm) as adsorbent and a 95:5 mixture of chloroform and acetone as eluent. The fractions containing solely the pure product [R_{f} (5)=0.7-08] were pooled and evaporated at 2.0-2.5 kPa. The oily residue was triturated with diethyl ether, filtered, washed with diethyl ether and dried. Yield, 8.34 g (88.5%) of pure benzyloxycarbonyl-D-cyclohexylglycine 2,4,5-trichlorophenyl ester.

### Step B: Synthesis of benzyloxycarbonyl-N-methyl-D-cyclohexylglycyl-L-azetidine-2-carboxylic Acid

L-Azetidine-2-carboxylic acid (1.01 g, 10 mM) and triethylamine (1.4 ml, 10 mM) were added to a solution of benzyloxycarbonyl-D-cyclohexylglycine 2,4,5-trichlorophenyl ester (5.18 g, 11 mM, from Example 4, Step A) in 10 ml pyridine. After stirring overnight the reaction mixture was evaporated under reduced pressure, and the residue was dissolved in 50 ml 5% NaHCO₃ and 50 ml diethyl ether. The organic phase was washed with water and the combined aqueous phases were washed with diethyl ether, acidified with 1 M KHSO₄ to pH 3 then extracted with 3×50 ml ethyl acetate. The ethyl acetate extracts were combined, washed with water to neutrality, dried over anhydrous Na₂SO₄, and evaporated at 2.0-2.5 kPa.

The evaporation residue is benzyloxycarbonyl-D-cyclohexylglycyl-L-azetidine-2-carboxylic acid that was dissolved in 10 ml tetrahydrofurane and combined with 5.0 ml (80 mM) iodomethane and cooled to 0° C. To this solution 1.2 g (30 mM) of sodium hydride 60% was added and the reaction mixture was stirred at RT overnight. Excess sodium hydride was decomposed by the careful addition of a 0.4 ml of water. The quenched reaction mixture was concentrated to about 10 ml under reduced pressure at a temperature below 30° C. The residue was diluted with 15 ml water and 10 ml of t-butyl methyl ether. The phases were separated, and the aqueous phase was washed again with 10 ml of t-butyl methyl ether. The aqueous product phase was combined with 15 ml of ethyl acetate and adjusted to pH 2.2 with 3M sulfuric acid solution. The phases were separated, and the aqueous phase was back-extracted with 10 ml of ethyl acetate. The combined organic phase was washed with 15 ml of a 5% sodium thiosulfate solution. The phases were separated, and the organic phase was evaporated pressure below 40° C. The oily residue is 3.75 g of benzyloxycarbonyl-N-methyl-D-cyclohexylglycyl-L-azetidine-2-carboxylic acid (9.65 mM, R_{f} (7)=0.85-0.95), which was dissolved in 9.65 ml tetrahydrofuran, and held for use in Step 1 of Example 4.

FAB mass spectrum (403 M+H]⁺) confirmed the assumed structure.

### EXAMPLE 5

### Inhibition of Clotting by Peptidyl Arginals

The inhibitory effect on plasma coagulation was evaluated in the thrombin time (TT), activated partial thromboplastin time (APTT) and prothrombin time (PT) assays by using citrated human plasma as substrate [Bagdy, D. et. al.: Thromb. Haemostas. 67, 325 (1992)]. The TT assay measures the inhibition of a single step, the coagulation of fibrinogen on the action of exogenous thrombin (final concentration 2.5 NIH U/ml). Clotting of plasma in the APTT and PT assays was induced by recalcification. Endogenous thrombin generated could theoretically be present at a final concentration as high as 50 NIH U/ml (APTT, PT). These assays detect the sum of inhibitory actions on fibrin formation and thrombin generation, which includes several proteolytic reactions mediated by either thrombin or other enzymes, e.g. fXa. Anticlotting activity is expressed in CT₂, which is the concentration (nM) required to double the clotting time.

TABLE 1
Inhibiting activities of the compounds of formula (I) of the
invention (1-4), the parent compound, Efegatran (C)
and further related peptidyl arginals (C1, C2) on plasma
coagulation (Column A), clot-bound thrombin and factor
Xa (Column B) and fibrinolytic enzymes (Column C)

| | | | | | B | | | | |
|---|---|---|---|---|---|---|---|---|---|
| Peptidyl arginals: | | A | | | IC₅₀ (nM) | | C | | |
| Xaa-Xbb-Arg-H^{a} | | CT₂ (nM)^{b} | | | | Factor | LA₅₀, μM^{c} | | |
| No. | Xaa-Xbb | TT | APTT | PT | Thrombin | Xa | PL | tPA | UK |
| 1 | Eoc-D-cHpa- | 147 | 331 | 1839 | 103 | 118 | 18 | 10 | 14 |
| | Pro | | | | | | | | |
| 2 | N-Me-D-cHpa- | 118 | 315 | 876 | 93 | 102 | 16 | 14 | 18 |
| | Pro | | | | | | | | |
| 3 | D-cHla-Pro | 86 | 281 | 1082 | 95 | 117 | 21 | 10 | 85 |
| 4 | N-Me-D-Chg- | 101 | 677 | 2921 | 245 | 457 | 32 | 12 | 16 |
| | Aze | | | | | | | | |
| C | D-MePhe-Pro | 87 | 622 | 2915 | 375 | 1000 | 54 | 132 | 82 |
| C1 | D-cHga-Pro | 120 | 333 | 1254 | 524 | 200 | 83 | 74 | 120 |
| C2 | Eoc-D-Cba-Pro | 114 | 349 | 1148 | 390 | 702 | 27 | 27 | 120 |

^{a}Abbreviations. Eoc = ethoxycarbonyl; cHpa = cycloheptylalanyl, cHla = cycloheptyllactyl; MePhe = N-methylphenylalanyl; cHga = cycloheptylglycolyl; Chg = cyclohexylglycyl.
^{b}CT₂ = concentration required for doubling the clotting times in the TT (thrombin time), APTT (activated partial thromboplastin time), and PT (prothrombin time) assays.
^{c}LA₅₀ = concentration required for the reduction of the lysed area to 50% of the control in the fibrin plate assay; PL = plasmin, tPA = tissue plasminogen activator, UK = urokinase.

Several of these compounds were superior to Efegatran (C, D-MePhe-Pro-Arg-H, the parent peptide arginal) in their ability to prolong clotting time (See Table 1). Column A of Table 1 presents the anticoagulant activities of the compounds of formula (I) of the invention (1-4) in comparison with those of Efegatran (C), a known anticoagulant [Bajusz, S. et al.: J. Med. Chem. 33, 1729 (1990); U.S. Pat. No. 4,703,036 (1982); Bagdy, D. et al.: Thromb. Haemostas. 67, 357 and 68, 125 (1992); Jackson, C. V. et al.: Clin. Appl. Thrombosis/Hemostasis 2, 258 (1996)] and further related peptidyl arginals (e.g. C1 and C2) [Bajusz, S. et al.: Bioorg. & Med. Chem. 3, 1079 (1995); U.S. Pat. No. 6,121,241 (2000); PCT Pub. No. WO97/46576]. The TT assay shows the new analogues closely as effective as efegatran in the inhibition of the thrombin-fibrinogen reaction. The APTT, on the other hand, indicates the new peptides are more effective than efegatran in the inhibition of the preceding, thrombin-generating steps of coagulation.

### EXAMPLE 6

### Inhibition of Thrombin and Factor Xa

Enzyme inhibition was examined in platelet-rich plasma clots by using chromogenic substrates, ie. Tos-Gly-Pro-Arg-pNA (S1) for thrombin and Moc-D-Chg-Gly-Arg-pNA (S2) for factor Xa, as published (Bajusz, S. et al.: PCT Pub. No. WO97/46576) briefly. The assays were carried out at room temperature in glass tubes and 96-well microtiter plates.

Solutions, (i) Buffer A: 0.1 M sodium phosphate/0.05 M NaCl (pH 8.5). (ii) Inhibitors: 0.1, 1.0 and 10 mg/ml solutions in buffer A containing 0.02% human albumin. (iii) Substrates: 1 mM of S1 and 2 mM of S2 in distilled water.

(b) Preparation of plasma clots. Platelet rich plasma samples (200 μl) placed in glass tubes were incubated at room temperature with 80 μl 40 mM CaCl₂ for 60 min. The clots were washed with 2 ml aliquots of saline (0.9% NaCl) with gentle agitation to remove unbound enzymes In case of successful washing the optical density of the reaction mixture of wash and S1 is less than 5% of the control. The plasma clot thus obtained was kept under saline in the tube until use.

(c) Assessment of the enzyme inhibition in the clots. After removal of saline, the plasma clot was incubated at 37° C. with 400 μl inhibitor (or buffer A as negative control) for 5 min. and with 100 μl substrate S1 or S3 for 30 min., then the reaction was stopped with 100 μl 50% acetic acid. 150 μl portions of the reaction mixtures were placed in the wells of a microtiter plate and read at 405 nm (ELISA READER 800, Bio-Tek Instruments Inc. Winooski, Vt., USA). IC₅₀ values were generated from the extinction data graphically.

Results are shown in Column B of Table 1. Compounds 1, 2, 3 and 4 are the only analogues that can surpass Efegatran in the inhibition of clot-bound thrombin but, in the inhibition of clot-bound factor Xa, each analog is better than Efegatran. Thus 1, 2, 3, and 4 are the most inhibitory for both dot bound enzymes.

### EXAMPLE 7

### Antifibrinolytic Activity

Inhibitory effects of the compounds of the invention on plasmin (PL) and plasmin generation by plasminogen (Plogen) activators, such as tissue plasminogen activator (tPA), and urokinase (UK) were examined by the fibrin plate assay. (See e.g., Bagdy, D., Barabás, E., Bajusz, S., and Széll, E., Thromb. Haemostas., 67:325-330 (1992); Barabás, E. Szell, E. and Bajusz, S, Blood Coagulation and Fibrinolysis, 4: 243 (1993)) The results are shown in Table 1, Column C. With a few exceptions the analogues are somewhat more inhibitory than Efegatran against the three fibrinolytic enzymes. The exceptions are C1 against PL, and C1, C2 against UK, while 3 is almost equiactive with Efegatran against UK.

### EXAMPLE 8

### Rabbit Model for Disseminated Intravascular Coagulation

Compounds 1 and 3 of the invention and the other peptidyl arginals of Table 1 as well as C3 were investigated for their DIC-inhibiting activity in endotoxin (lipopolysaccharide, LPS) treated rabbits, as described [Scherer, M. U. et al.: Lab. Anim. Sci. 45, 538 (1995)]. The assay procedure lasted for 4 hours. Endotoxin was administered in doses of 80 and 40 μg/kg in i.v. bolus injection, at 0 and 120 min, respectively, while peptidyl arginals 1, 3, and C-C3 (0.25 and/or 0.5 mg/kg/h) were infused along the whole experiment. Control group of animals was treated with 0.9% saline. Hemostatic parameters were determined at 0, 120, and 240 min.

In spite of careful treatment, lethality occurred in 31%, most likely due to the high endotoxin-sensitivity of rabbits [Semerano, N. et al.: Int. J. Clin. Lab. Res 21, 214 (1992)].

TABLE 2
Effect of compounds 1 and 3 of the invention, the parent
compound Efegatran (C), further arginals (C1 and C2) and
heparin (H) on lethality of endotoxin-treated rabbits

| Lethality | |
|---|---|
| no. died/treated animals and % | |
| 2 hrs | 4 hrs |

| Agents^{a} | No. | % | No. | % |
|---|---|---|---|---|
| Salsol (0.9% NaCl) | 0/10 | 0 | 0/10 | 0 |
| Endotoxin | 0/13 | 0 | 4/13 | 31 |
| +1, 0.5 mg/kg/h | 0/12 | 0 | 2/12 | 17 |
| +1, 0.25 mg/kg/h | 0/19 | 0 | 3/19 | 16 |
| +3, 0.25 mg/kg/h | 0/22 | 0 | 4/22 | 18 |
| +C, 0.5 mg/kg/h | 1/15 | 7 | 6/15 | 40 |
| +C, 0.25 mg/kg/h | 1/14 | 7 | 5/14 | 36 |
| +C1, 0.5 mg/kg/h | 0/16 | 0 | 5/16 | 31 |
| +C2, 0.5 mg/kg/h | 0/12 | 0 | 5/12 | 42 |
| +C3, 0.5 mg/kg/h | 1/18 | 6 | 10/18 | 56 |
| +H, 100 U/kg/h | 0/19 | 0 | 7/19 | 37 |
| +H, 50 U/kg/h | 0/17 | 0 | 6/17 | 35 |

^{a}See Table 1 for the structures of peptidyl arginals 1, 3, C, C1 and C2, C3 = hPla-Pro-Arg-H wherein hPla = 2-hydroxy-4-phenylbutyric acid.

As data of Table 2 show, compounds 1 and 3 significantly reduce the lethality of endotoxin-treated rabbits, while the other anticoagulants either have no effect on lethality (C1) or cause some increase in lethality, the highest value, ˜1.8-fold, is obtained with C3. It is worth noting from the data of Table 1 that lethality reducing 1 and 3 are the most inhibitory against clot-bound thrombin as well as factor Xa, and also efficiently inhibit both plasma coagulation and the fibrinolytic enzymes, plasmin, and plasminogen activators.

### EXAMPLE 9

### Rat Model for Disseminated Intravascular Coagulation

Among the most serious consequences of DIC are fibrin deposition in various organs, blood cell changes, e.g. reduction of platelet count, and changes in fibrin degradation products. The effects of compound 1 of the invention and two control anticoagulants, Efegatran (C) and heparin (H) on such phenomena were examined in endotoxin-treated rats [Ford, A. J. and Longridge, D. J.: Br. J. Pharmacol. 110, Suppl. 131P (1993); Hasegawa, N.; et al.: Am. J. Resp. Crit. Care Med. 153, 1831 (1996); Dichneite, G. et al.: Thromb. Res. 77, 357 (1995)].

Male rats were treated with an i.v. bolus injection of 10 mg/kg endotoxin. It was followed by i.v. infusion of saline or the test compounds for four hours. Of compounds 1 and 3, 0.25 mg/kg was given as an initial bolus injection followed by an 0.25 mg/kg/h i.v. infusion for four hours. Heparin (H) was applied similarly, 50 IU/kg as an initial bolus injection followed by an 50 IU/kg/h i.v. infusion for four hours. Control group of animals was treated with 0.9% saline.

The deposition of ¹²⁵I-fibrin was investigated in selected organs (liver and kidney. ¹²⁵I-fibrinogen was injected 30 min. prior to endotoxin injection. The radioactivities in the tissue samples were measured in a gamma counter (Wallac Wizard 1470). Microthrombi formation in the organs was assessed using the ratio of organ ¹²⁵I activity to injected total ¹²⁵I activity, defined as the microthrombi index. Changes in this parameter are expressed in percent compared to saline group.

Number of platelet count was determined in an automatic appliance (Sysmex F-800) and related to control values.

Determination of FDP (fibrin degradation products) by Aggristin (Ristocetin) precipitation assay. Animals were killed four hours after endotoxin administration.

Findings are summarized in Table 3.

TABLE 3
Effects of compound 1 of the invention, the parent peptide,
Efegatran (C), and heparin (H) on ¹²⁵I-fibrin
deposition, and on changes in platelet count and fibrin
degradation products (FDP) in endotoxin-treated rats

| | ¹²⁵I-fibrin | | Change in | |
|---|---|---|---|---|
| | deposition | | platelet | Change |
| Agents | Liver | Kidney | Count | in FDP |
| Endotoxin, 10 mg/kg^{a} | 36% | 36% | 62% | 2.56 |
| +1, 0.25 mg/kg/h^{b} | 13% | 26% | 48% | 1.66 |
| +C, 0.25 mg/kg/h^{b} | 30% | 33% | 52% | 1.94 |
| +H, 50 NIH U/kg/h^{b} | 22% | 28% | 43% | 2.37 |

^{a}I.V. bolus injection.
^{b}I.V. bolus injection + I.V. infusion.

Data of Table 3 indicate the DIC-inhibiting potential of 1 was more pronounced than that of either heparin or Efegatran.

### EXAMPLE 10

### Examination of the Survival in a Rat Model for Disseminated Intravascular Coagulation

Male rats were treated with an i.v. bolus of 30 mg/kg LPS (endotoxin). Peptides in doses of 0.5 and/or 0.75, 1.0 and 1.5 mg/kg were given as an initial bolus injection followed by the infusion for eight hours immediately after LPS administration. Mortality was recorded at 4, 5, 6, 7, 8 hours post LPS.

Data of Table 4 indicate that LPS treatment reduced the survival rate of the rats to 20% by the end of experiment (8 hours). All investigated new peptidyl arginals prolonged the survival time and reduced mortality compared to LPS group. Compounds 1, 2, 3, and 4 were more effective than reference C.

TABLE 4
Effects of compounds 1, 2, 3, and 4 of the invention and
of parent compound C on lethality of LPS-treated rats

| | Survival rate % | | | | | | | | | | | | | | | |
|---|---|---|---|---|---|---|---|---|---|---|---|---|---|---|---|---|
| Time | Control | C, mg/kg | | | 1, mg/kg | | | 2, mg/kg | | | 3, mg/kg | | | 4, mg/kg | | |
| (hours) | 0 | 0.75 | 1.0 | 1.5 | 0.75 | 1.0 | 1.5 | 0.75 | 1.0 | 1.5 | 0.5 | 0.75 | 1.5 | 0.5 | 0.75 | 1.5 |
| 0 | 100 | 100 | 100 | 100 | 100 | 100 | 100 | 100 | 100 | 100 | 100 | 100 | 100 | 100 | 100 | 100 |
| 4 | 76 | 93 | 83 | 100 | 100 | 100 | 100 | 86 | 100 | 100 | 100 | 100 | 100 | 93 | 100 | 100 |
| 5 | 72 | 73 | 83 | 100 | 100 | 100 | 100 | 78 | 100 | 100 | 100 | 100 | 100 | 87 | 100 | 100 |
| 6 | 37 | 67 | 66 | 100 | 100 | 100 | 100 | 57 | 100 | 100 | 100 | 100 | 100 | 87 | 100 | 100 |
| 7 | 24 | 33 | 58 | 92 | 90 | 90 | 100 | 57 | 90 | 100 | 100 | 100 | 100 | 87 | 92 | 100 |
| 8 | 20 | 27 | 50 | 92 | 70 | 90 | 100 | 50 | 90 | 100 | 80 | 92 | 92 | 80 | 85 | 100 |

Male rats were treated with an i.v. bolus of 30 mg/kg LPS.
Test compounds were given as an initial bolus followed by an i.v. infusion for eight hours immediately after the administration of LPS.
Mortality was recorded at 4, 5, 6, 7, and 8 hours post LPS.

1. A compound having the formula (I)
Xaa-Xbb-Arg-H (I)
wherein Xaa represents an alpha-substituted carbonic acid residue of formula (II)
Q-CH(R)—CO (II)
wherein Q represents a 1-3 carbon alkyloxycarbonylamino group, a methylamino group, or a hydroxyl group, and R represents a 7-9 carbon cycloalkyl ethyl group, a 1-adamantylmethyl group, or a 5-7 carbon cycloalkyl group, and Xbb represents an L-proline or L-azetidine-2-carboxylic acid residue, and the acid-addition salts thereof formed with organic or inorganic acid.

2. A compound having the structure 1: 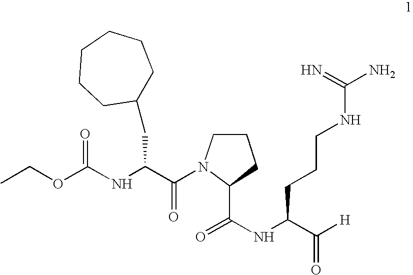
and the acid-addition salts thereof.

3. A compound having the structure 2: 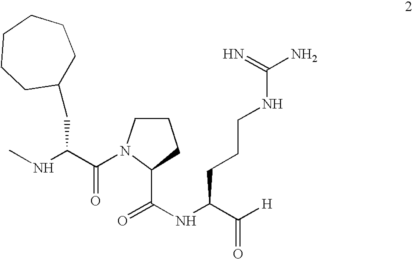
and the acid-addition salts thereof.

4. A compound having the structure 3: 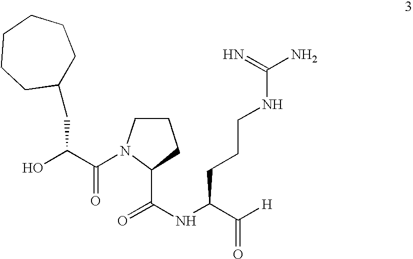
and the acid-addition salts thereof.

5. A compound having the structure 4: 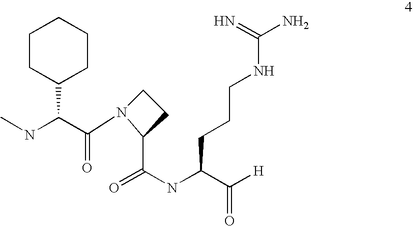
and the acid-addition salts thereof.

6. A pharmaceutical formulation comprising a compound according to claim 1.

7. A pharmaceutical formulation comprising a compound according to claim 2.

8. A pharmaceutical formulation comprising a compound according to claim 3.

9. A pharmaceutical formulation comprising a compound according to claim 4.

10. A pharmaceutical formulation comprising a compound according to claim 5.

11. The compound ethoxycarbonyl-D-cycloheptylalanyl-L-prolyl-N^{G}-benzyloxycarbonyl-L-arginine aldehyde.

12. The compound tetrahydropyranyl-D-cycloheptyl-lactyl-L-prolyl-N^{G}-benzyloxycarbonyl-L-arginine aldehyde.

13. The compound benzyloxycarbonyl-N-methyl-D-cycloheptyl-alanyl-L-proiyl-N^{G}-benzyloxycarbonyl-L-arginine aldehyde.

14. The compound N-methyl-D-cyclohexylglycyl-L-azetidine-2-carbonyl-L-arginine aldehyde.

15. The pharmaceutical formulation of any of claims 6-10 wherein said formulation comprises a tablet, capsule, powder, pill, dragée, granulate, solution, infusion, suppository, plaster or ointment.

16. A method for treating a patient having disseminated intravascular coagulation, the method comprising administering to the patient a peptidyl arginal having inhibiting action on clot bound thrombin, factor Xa, plasmin, and plasminogen activators.

17. A method for treating a patient having disseminated intravascular coagulation, the method comprising administering to the patient a peptidyl arginal having the formula (I)
Xaa-Xbb-Arg-H (I)
wherein Xaa represents an alpha-substituted carbonic acid residue of formula (II)
Q-CH(R)—CO (II)
wherein Q represents a 1-3 carbon alkyloxycarbonylamino group, a methylamino group, or a hydroxyl group, and R represents a 6-9 carbon cycloalkylmethyl group, a 1-adamantylmethyl group, or a 5-7 carbon cycloalkyl group, and Xbb represents an L-proline or L-azetidine-2-carboxylic acid residue, or a pharmaceutically acceptable acid-addition salts thereof.

18. A method for treating a patient having disseminated intravascular coagulation, the method comprising administering to the patient a peptidyl arginal having the structure 1: 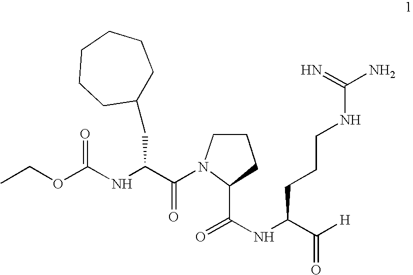
or a pharmaceutically acceptable acid-addition salts thereof.

19. A method for treating a patient having disseminated intravascular coagulation, the method comprising administering to the patient a peptidyl arginal having the structure 2: 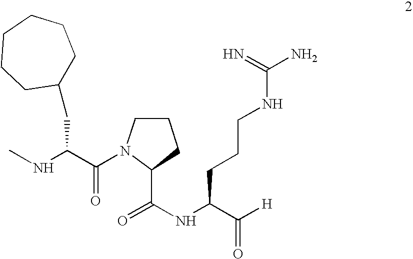
or a pharmaceutically acceptable acid-addition salts thereof.

20. A method for treating a patient having disseminated intravascular coagulation, the method comprising administering to the patient a peptidyl arginal having the structure 3: 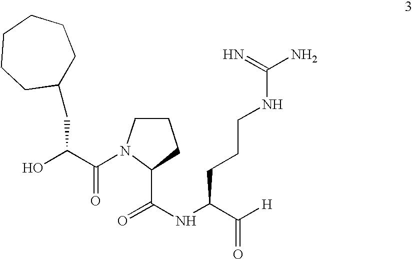
or pharmaceutically acceptable acid-addition salts thereof.

21. A method for treating a patient having disseminated intravascular coagulation, the method comprising administering to the patient a peptidyl arginal having the structure 4: 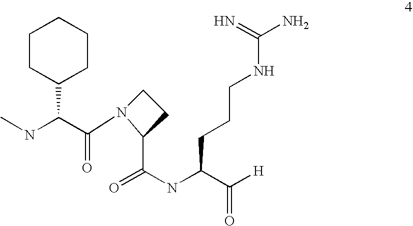
or pharmaceutically acceptable acid-addition salts thereof.

22. The method according to claim 1, wherein the patient is administered from about 0.1 mg to about 50 mg/kg of a peptidyl arginal based on patient body weight.

23. The method according to claim 1, wherein the patient is administered a peptidyl arginal at a dosage sufficient to attain a blood level of peptidyl arginals from about 6 μM to about 100 μM.

24. The method according to claims 7 or 8, wherein the peptidyl arginal administration is simultaneous or sequential.
